SEQUENCE LISTING (4 sequences)

SEQ ID NO: 1 - protein, 13 aa, Artificial sequence
  synthetic
LCTPSRAALLTGR

SEQ ID NO: 2 - protein, 6 aa, Artificial sequence
  synthetic
LCTPSR

SEQ ID NO: 3 - protein, 6 aa, Artificial sequence
  synthetic
  X (2)..(2): X is cysteine or serine
LXTPSR

SEQ ID NO: 4 - protein, 6 aa, Artificial sequence
  synthetic
  X (2)..(2): X is a 2-formylglycine residue
LXTPSR

(19) United States
(12) Patent Application Publication
(10) Pub. No.: US 2024/0209047 A1
(43) Pub. Date: Jun. 27, 2024

(54) GROWTH FACTOR CONJUGATES

(71) Applicants: Vijay Krishna Raghunathan, Houston, TX (US); Christopher J. Murphy, Madison, WI (US); Nicholas L. Abbott, Madison, WI (US)

(72) Inventors: Vijay Krishna Raghunathan, Houston, TX (US); Christopher J. Murphy, Madison, WI (US); Nicholas L. Abbott, Madison, WI (US)

(21) Appl. No.: 18/287,249

(22) PCT Filed: Apr. 20, 2022
(86) PCT No.: PCT/US2022/025493

§ 371 Date: Oct. 17, 2023

Related U.S. Application Data
(60) Provisional application No. 63/176,965, filed on Apr. 20, 2021.

(51) Int. Cl. C07K 14/475; A61K 47/54; A61P 17/02; A61K 38/00

(52) U.S. Cl. CPC C07K 14/475; A61K 47/54; A61P 17/02; A61K 38/00

(57) ABSTRACT

The present invention relates to the treatment of chronic wounds, and in particular to treatment of chronic wounds by via the use of growth factors conjugates that are immobilized in the wound. Specifically, the growth factor (GF) conjugates comprising a GF molecule covalently modified with a chemical moiety comprising a reactive functionality. Further disclosed arm GF molecules that are used for the conjugates and the chronic wounds can be treated using the GF conjugates.

### CROSS-REFERENCE TO RELATED APPLICATION

This application claims priority to and the benefit of U.S. Provisional Patent Application No. 63/176,965, filed Apr. 20, 2021. The entire contents of which are hereby incorporated by reference in its entirety.

### STATEMENT REGARDING FEDERALLY SPONSORED RESEARCH OR DEVELOPMENT

This invention was made with government support under 290 RC2AR058971 awarded by the National Institute of Health, and P30EY12576 and K08EY030950 from the National Eye Institute. The government has certain rights in the invention.

### FIELD OF THE INVENTION

The present invention relates to the treatment of chronic wounds, and in particular to treatment of chronic wounds by via the use of growth factors conjugates that are immobilized in the wound.

### BACKGROUND OF THE INVENTION

Chronic wounds encompass a variety of conditions such as venous leg ulcers, pressure ulcers, vascular insufficiency, and diabetic foot ulcers to name a few. They are a significant burden to patients with profound effects on their quality of life and on healthcare costs worldwide. In 2009 in the United States, chronic wounds were reported to affect around 6.5 million patients with an excess of $25 billion spent annually on their treatment (Marquez et al., 2002, Sen et al., 2009). More recently, the American Diabetes Association, in 2013, estimated that the total economic burden for ‘diagnosed diabetes’ in the US alone was a staggering $245 billion attributed to increasing cost of care with rising prevalence, demographics, and changes in treatment access, modalities and comorbidities (2013, American Diabetes, 2013). These costs are only expected to rise and suggest that cost-effective treatment and care would significantly alleviate future economic burden.

Wound healing is a complex process with highly integrated and overlapping phases of hemostasis, inflammation, epithelialization, formation of granulation tissue, neovascularization, wound contraction, and extracellular matrix (ECM) reorganization (FIG. 1). A critical clinical endpoint considered by the US Food and Drug Administration for wound closure is re-epithelialization (Gottrup et al., 2010, Maderal et al., 2012, Robson et al., 1999, Stromberg et al., 1994), a process that can be prolonged in pathologic wounds (e.g. diabetes, venous stasis, denervation) as well as extensive wounds associated with trauma and burns. Chronic wounds heal differently from acute wounds and as such require different treatment modalities. They have a prolonged inflammatory phase, are susceptible to infections, and are unable to respond to repair stimuli from within the wound bed. This is largely because, cells residing in chronic wounds have a lower density of growth factor receptors and lower mitogenic potential resulting in impaired response to environmental cues (Lerman et al., 2003, Loot et al., 2002, Raffetto et al., 2001, Seidman et al., 2003, Vasquez et al., 2004).

Growth factors are intrinsic elements that are critical to the orchestration of events throughout the wound healing process. Within wounds, cytoactive factors are present both in soluble form and as bound to the extracellular matrix (ECM) (Hynes, 2009). These factors when confined to the ECM, albeit at low surface concentrations, provide localized i5 interactions to numerous surface receptors of cells to elicit a cellular response. To account for mass transport across the space of the fluid bathing the cells of the wound surface, soluble factors may be required at greater concentrations than intrinsically immobilized factors to elicit a response and have shorter resident times. Numerous soluble cytoactive factors have been investigated for their ability to improve wound healing outcomes with platelet-derived growth factor (PDGF), fibroblast growth factor (FGF), vascular endothelial growth factor (VEGF), insulin-like growth factor (IGF), transforming growth factor β (TGFβ) and epidermal growth factor (EGF) (Barrientos et al., 2008, Falanga) being among the most intensively studied. While numerous growth factors promote proliferation and migration, repeated topical administration of cytoactive factors in large doses may lead to toxic adverse events including tumorigenicity (Abramovitch et al., 1999). In fact, over the past two decades only one cytoactive growth factor, recombinant human PDGF (rhPDGF-BB; Regranex, Healthpoint Ltd, Ft. Worth, Tx) has been approved by the FDA for treatment of chronic wounds (Wieman et al., 1998) which, notably, is accompanied with a ‘black box’ warning for potentially increasing neoplastic transformation systemically.

Thus, there is an urgent need for viable treatments to promote healing of chronic wounds.

### SUMMARY OF THE INVENTION

The present invention relates to the treatment of chronic wounds, and in particular to treatment of chronic wounds by via the use of growth factors conjugates that are immobilized in the wound.

In some preferred embodiments, the present invention provides a growth factor (GF) conjugate comprising a GF molecule covalently modified with a chemical moiety comprising a reactive functionality. In some preferred embodiments, the GF molecule is recombinant. In some preferred embodiments, the GF molecule is a human GF molecule. In some preferred embodiments, the GF molecule is a non-human EGF molecule. In some preferred embodiments, the GF molecule is wild-type. In some preferred embodiments, the GF molecule is non-wildtype. In some preferred embodiments, the GF molecule comprises one or more mutations as compared to wild-type GF. In some preferred embodiments, the mutations are substitution mutations. In some preferred embodiments, the GF molecule comprises one or more additional amino acids at the N- or C-terminal of the GF molecule.

In some preferred embodiments, the GF molecule comprises a genetically encoded aldehyde tag. In some preferred embodiments, the genetically encoded aldehyde tag has a sequence selected from the group consisting of

(SEQ ID NO: 1)
LCTPSRAALLTGR (ALD_{13;})
and
(SEQ ID NO: 2)
LCTPSR (ALD₆).

In some preferred embodiments, the GF molecule is selected from the group consisting of an Epidermal Growth Factor (EGF) molecule, a Vascular Endothelial Growth Factor (VEGF) molecule, a Platelet-Derived Growth Factor (PDGF) molecule, a Fibroblast Growth Factor (FGF) molecule, a Keratinocyte Growth Factor (KGF) molecule, a Transforming Growth Factor Beta (TGF-β) molecule, a Granulocyte-Macrophage Colony-Stimulating Factor (GM-CSF) molecule, an Insulin-Like Growth Factor I (IGF-I) molecule, an Insulin-Like Growth Factor II (IGF-II) molecule, Nerve Growth Factor (NGF).

In some preferred embodiments, the reactive functionality is selected from the group consisting of a sulfhydryl reactive functionality, an amine reactive functionality, a carbonyl reactive functionality, and a carboxyl reactive functionality. In some preferred embodiments, the chemical moiety is covalently attached to the GF molecule at a chemical group in the GF molecule selected from the group consisting of an amine group, a sulfhydryl group, a carbonyl group, and a carboxyl group.

In some preferred embodiments, the present invention provides methods of treating a chronic wound in a subject in need thereof comprising: applying a conjugate as described above to the chronic wound under conditions such that the conjugate covalently binds to a target group in the wound that is reactive with the reactive functionality. In some preferred embodiments, the subject is a human subject. In some preferred embodiments, the subject is a non-human subject. In some preferred embodiments, the chronic would is selected form the group consisting diabetic ulcers, pressure ulcers, traumatic ulcers, venous stasis ulcers, venous leg ulcers, and arterial ulcers. In some preferred embodiments, the chronic wound has a wound surface and the conjugate is applied to the wound surface in an amount sufficient to cover the wound surface with from 0.1 to 20 pg of the conjugate per mm² of the wound surface. In some preferred embodiments, the conjugate is applied every 24 to 72 hours. In some preferred embodiments, the conjugate is applied every 36 to 60 hours. In some preferred embodiments, the conjugate is applied until more than 50% of the wound is re-epithelialized. In some preferred embodiments, the conjugate is applied until more than 75% of the wound is re-epithelialized. In some preferred embodiments, the conjugate is applied until more than 90% of the wound is re-epithelialized. In some preferred embodiments, the conjugate is applied until more than 95% of the wound is re-epithelialized. In some preferred embodiments, the conjugate is applied to the wound from about 3 to about 30 times. In some preferred embodiments, the conjugate is applied to the wound from about 3 to about 20 times. In some preferred embodiments, the conjugate is applied to the wound from about 5 to about 15 times. In some preferred embodiments, the conjugate is provided in or delivered via a polymeric matrix.

In some preferred embodiments, the present invention provides a conjugate as described above for use in treating a chronic wound in a subject in need thereof. In some preferred embodiments, the chronic would is selected form the group consisting diabetic ulcers, pressure ulcers, traumatic ulcers, venous stasis ulcers, venous leg ulcers and arterial ulcers. In some preferred embodiments, the conjugate is provided in or delivered via a polymeric matrix.

In some preferred embodiments, the present invention provides a device comprising the conjugate as described above in a polymeric matrix.

### BRIEF DESCRIPTION OF THE DRAWINGS

FIG. 1. Schematic of events during wound repair. Hemostasis beings on the onset of injury and is followed by inflammatory phase. During inflammation, debris and pathogens are removed, accompanied by secretion of growth factors to facilitate wound repair. This is followed by the proliferative phase which includes the formation of a fibrin clot, re-epithelialization, and contraction of wound margin. The last phase involves remodeling where the matrix reorganizes and wound is fully closed.

FIG. 2-B. (A) Topically applied soluble EGF did not promote epithelial closure. Percentage wound closure in mice with splinted full thickness wounds (8 mm diameter) treated without any treatment (NoTx), or with either soluble EGF (sEGF), or vehicle over 11 days. No significant effect of group or interaction between group and time was observed (P>0.24, repeated measures ANOVA). No significant differences between groups were observed for any time point (p>0.05, Kruskal-Wallis test). (B) sEGF is rapidly removed from wound surface. Persistence of sEGF in the wound bed as determined by ELISA specific to anti-human EGF. Amount of EGF measured on the wound bed is expressed as mass per mm² area of the tissue.

FIG. 3A-D. Crosslinking EGF to biological surfaces using thiol chemistry. (A) Representative schematic of the chemistry employed for covalent immobilization of EGF onto wound bed.

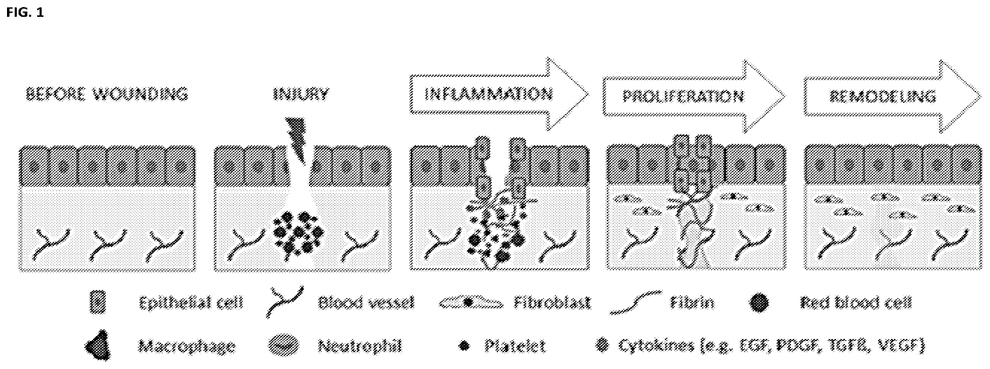

Covalently modified growth factors (e.g., EGF) using heterobifuctional crosslinkers are linked to disulfide groups on the wound surface. Free sulfhydryl groups on the wound surface are exposed using a potent yet non-toxic dose of a reducing agent. (B) Cross linking increases persistence of EGF on the wound surface in vivo. Persistence of crosslinked EGF (CxEGF) and reactive-moiety quenched EGF (qEGF) in the wound bed as determined by ELISA specific to anti-human EGF. Amount of EGF measured on the wound bed is expressed as mass per area of the tissue. (C) Covalent immobilization of EGF strongly promotes proliferation in vitro. Human 433 immortalized kertinocytes (HaCaT) cells were cultured in growth medium containing 1% (v/v) serum (FBS). The proliferation rates are expressed as % control when cultured on surfaces [trimethoxy(propyl)silane (TPS) or 3-mercaptopropyl trimethoxysilane (3-MPTS)] immobilized with EGF. As positive control, cells were also cultured in the growth medium containing 10% 437 (v/v) FBS. ***p<0.001, one-way ANOVA followed by Dunnett's multiple comparison test compared with control cultures (cells cultured in media with 1% serum). (D) Changes in contact angle and thickness of silanized surfaces after immobilization with malemide-conjugated PEG demonstrating proof of covalent linkage chemistry.

FIG. 4A-C. Covalent immobilization of EGF in diabetic wound beds promotes epithelial wound closure. (A) Images of representative animals from the groups of CxEGF-6x, sEGF, and NoTx which were taken on days 0, 6, 11. (i) CxEGF-6x, day 0; (ii) CxEGF-6x, day 6; (iii) CxEGF-6x, day 11; (iv) sEGF, day 0; (v) sEGF, day 6; (vi) sEGF, day 11. (vii) NoTx, day 0; (viii) NoTx, day 6; (ix) NoTx, day 11. (B) Crosslinked EGF treated 6 times over 11 days (CxEGF-6x) group had significantly improved wound healing compared to the soluble EGF (sEGF) delivered topically twice daily, no treatment (No Tx), and quenched EGF (qEGF-6x) groups. The CxEGF-6x group had a significantly higher percentage wound closure compared to the NoTx group (P<0.01). The CxEGF-6x group had a significantly higher percentage wound closure compared to the qEGF group (P<0.01). The NoTx group had a significantly higher percentage wound closure compared to the qEGF group (P<0.04)]. (C) The groups of CxEGF-6x and CxEGF-3x had significantly higher wound closure rate (decrease in wound radius per day) compared to the rest of the groups including the sEGF group (P<0.05).

FIG. 5A-C. Histopathological evaluations of a wound bed from a db/db mouse treated with/without crosslinked EGF over 11 days upon wounding. Scale Bars=500 μm. (A) Measurement of epithelial gap (blue line) and reepithelialization (red line). The epithelial gap was defined as the distance between the advancing edges of keratinocyte migration measured in millimeters. Length of reepithelialization was defined as the length of the layer of proliferating keratinocytes covering the wound area. This value was obtained by measuring s the distance between the free edge of the keratinocyte layer and the base where the cells were still associated with native, non-affected dermal tissue. The final value was the sum of distance in millimeters of both sides. H&E staining. Graphs indicate the mean f standard deviation of epithelial gap/re-epithelialized measure on surface wounds after 11 days with or without treatments. (B) Measurement of the fibrovascular dermal proliferation in the wound bed. Using picrosirius red stain under polarized light the bright collagen fibers of the wound bed are highlighted and automatically measured by the software. The final data is expressed as a percentage of outlined wound area comprised of collagen. Graphs indicate the mean f standard deviation of % collagen measured in surface wounds after 11 days with or without treatments. (C) Cross linked EGF elicited less inflammation. The inflammatory response was assessed using a semi-quantitative scoring system ranging from 0 to 4 where 0 indicates no inflammation, 1 indicates 0-25% of the wound area affected, 2 indicates 25-50% of the wound area affected, 3 indicates 50-75% of the wound area affected, and 4 indicates 75% of the wound area affected. H&E staining. Graphs indicate the mean±standard deviation of inflammation score. *p<0.05 compared with qEGF-6x, #p<0.05 compared with NoTx (t-test/Mann-Whitney rank sum test).

FIG. 6. Wound closure rate per applied EGF amount in the CxEGF-6x group was about 50 times higher than that in the sEGF group, which was statistically significant (kp<0.001). This indicates that significantly less amount of test article can be utilized with improved therapeutic effect.

FIG. 7A-C. Histopathological evaluations of a wound bed from a db/db mouse treated with/without topically applied EGF (twice daily) over 11 days upon wounding. Scale Bars=500 μm. (A) Measurement of epithelial gap (blue line) and reepithelialization (red line). The epithelial gap was defined as the distance between the advancing edges of keratinocyte migration measured in millimeters. Length of reepithelialization was defined as the length of the layer of proliferating keratinocytes covering the wound area. This value was obtained by measuring the distance between the free edge of the keratinocyte layer and the base where the cells were still associated with native, non-affected dermal tissue. The final value was the sum of distance in millimeters of both sides. H&E staining. Graphs indicate the mean f standard deviation of epithelial gap/re-epithelialized measure on surface wounds after 11 days with or without treatments. (B) Measurement of the fibrovascular dermal proliferation in the wound bed. Using picrosirius red stain under polarized light the bright collagen fibers of the wound bed are highlighted and automatically measured by the software. The final data is expressed as a percentage of outlined wound area comprised of collagen. Graphs indicate the mean f standard deviation of % collagen measured in surface wounds after 11 days with or without treatments. (C) Cross linked EGF elicited less inflammation. The inflammatory response was assessed using a semiquantitative scoring system ranging from 0 to 4 where 0 indicates no inflammation, 1 indicates 0-25% of the wound area affected, 2 indicates 25-50% of the wound area affected, 3 indicates 50-75% of the wound area affected, and 4 indicates 75% of the wound area affected.

FIG. 8. In vitro proliferation rates of spontaneously immortalized human keratinocytes (HaCaT cells), cultured in growth medium containing 1% (v/v) serum (FBS), treated with 0-80 ng/ml soluble EGF. Results are mean f standard deviation, ***p<0.001 (ANOVA followed by Dunnett's multiple comparison test) compared with control cultures. Soluble EGF increased cell proliferation.

FIG. 9A-C. Heterobifunctional crosslinker sulfo-SMCC significantly improves attachment of PLGA nanospheres (NS) on Gammagraft. Fluorescent micrographs of Gammagraft samples treated with (A) unmodified NS, and (B) sulfo-SMCC activated NS. (C) Pre-activation of the NS with sulfo-SMCC improved their attachment to Gammagraft by 83%. n=6 samples per treatment group, 4 images per sample; *** p<0.001.

FIG. 10. HaCaT and HMVEC viability is differentially affected by the hydrolyzed state and concentration of (A) sulfo-SMCC or (B) TCEP. In vitro screening of HaCaT (♦) and HMVEC (▪) viability by calcein-AM assay following 1 h exposure to fresh (light pink ♦ and light blue ▪), and hydrolyzed (dark pink ♦ and dark blue ▪), solutions of in DPBS, at a concentration range of 0.001 μM to 10000 μM (1 nM to 10 mM). Maximum concentration of sulfo-SMCC was 1 mM due to lower solubility in DPBS. For HaCaTs, n=10 for each state and concentration. For HMVECs, n=5 for each state and concentration. Error bars are plus/minus SEM.

FIG. 11. Percentage distribution of inflammatory cells in wound bed of diabetic mice at the end of 11 days. No statistically significant differences in (A) F-480 positive macrophages or (B) GR1 positive granulocytes were detected in wound bed from a db/db mouse treated with/without crosslinked EGF after 11 days upon wounding. Results are mean f standard deviation of the ratio of positive cells to total number of cells in the wound bed.

### DETAILED DESCRIPTION OF THE INVENTION

Wound healing is an enormously complex process that requires the simultaneous cellular integration of multiple soluble biochemical cues, as well as cellular responses to the intrinsic chemistry associated with the matrix of the wound space. Specifically, re-epithelialization of wounds is a critical element of wound closure. Growth factors have been used in combination with conventional wound management to promote closure but the method of delivery has been limited to topical application of ointment formulations. Growth factors delivered in this way have short resident times in wounds and have met with limited success. Further, controlled release systems are complex, externally applied devices, and inherently difficult to fabricate. Thus, retention of bio-active growth factors at the wound interface would be significantly beneficial clinically.

Here, we demonstrate that methods used to covalently immobilize proteins on synthetic materials can be extended to immobilize growth factors such as epidermal growth factor (EGF) onto the wound beds (wound interface) and accelerate wound closure in genetically diabetic mice with impaired healing. This study provides a new paradigm for changing the chemistry of wound bed, enabling tethering of growth factors to enhance wound closure, and in particular re-epithelialization of the wound. Major advantages of the methods described herein are the enhanced retention times, requirement for fewer dosing, and significantly less amount of growth factor required to enhance wound healing compared with topical application.

In some preferred embodiments, the present invention provides growth factor conjugates for use in application to the surface of a wound. By “conjugate” is meant a first moiety that is stably associated with a second moiety. By “stably associated” is meant that a moiety is bound to another moiety or structure under standard conditions. In certain embodiments, the first and second moieties are bound to each other through one or more covalent bonds. In some preferred embodiments, the growth factor conjugate comprises a growth factor modified with a chemical moiety that comprises a reactive functionality. In some preferred embodiments, the growth factor conjugates of the present invention may be represented by the following formula:

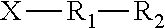

wherein X is a growth factor molecule, R₁ is a chemical moiety covalently attached to the growth factor molecule and R₂ is a reactive functionality. Thus, the first moiety in the conjugate is the growth factor (X) which is stably associated with the second moiety R₁ which presents the reactive functionality R₂.

The present invention is not limited to the use of any particular growth factor molecule. Suitable growth factor molecules are known in the art and include, but are not limited to Epidermal Growth Factor (EGF) and associated EGF-family growth factors, Vascular Endothelial Growth Factor (VEGF) and associated VEGF-family growth factors, Platelet-Derived Growth Factor (PDGF), acidic and basic Fibroblast Growth Factor (FGF) and associated FGF-family growth factors, Keratinocyte Growth Factor (KGF), Transforming Growth Factor Beta (TGF-β) and associated TGF-β-family growth factors, Granulocyte-Macrophage Colony-Stimulating Factor (GM-CSF), Insulin-Like Growth Factor I (IGF-I), Insulin-Like Growth Factor II (IGF-II), and Nerve Growth Factor (NGF). It will be recognized in the art that the amino acid sequences for these growth factors for numerous species are known in the art and that many of the growth factors are commercially available.

The EGF family of growth factors includes over a dozen proteins. EGF is the founding member of the EGF-family of proteins. Members of this protein family have highly similar structural and functional characteristics. Besides EGF itself other family members include: Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4). All family members contain one or more repeats of the conserved amino acid sequence: CX₇CX₄₋₅CX₁₀₋₁₃CXCX₈GXRC wherein C is cysteine, G is glycine, R is arginine, and X represents any amino acid. This sequence contains six cysteine residues that form three intramolecular disulfide bonds. Disulfide bond formation generates three structural loops that are essential for high-affinity binding between members of the EGF-family and their cell-surface receptors. This subset of proteins has been extensively studied and is known to facilitate re-epithelialization by stimulating the proliferation and migration of keratinocytes. Secondarily, the EGF family of proteins is responsible for increasing the tensile strength of new skin. EGF proteins are secreted by fibroblasts, platelets, and macrophages and localize throughout the epidermis, particularly in the basal layer.

Within this family of growth factors, EGF has experienced the greatest use in human subjects. EGF has been used to supplement the healing of skin grafts following partial-thickness burns. Other members of the EGF family that have known roles in wound healing are HB-EGF and TGF-α. In animal studies, HB-EGF was transiently upregulated 2 to 4 days after wounding, indicating a role for this protein in early healing. Moreover, application of HB-EGF to full-thickness wounds in mice increased proliferation and migration of keratinocytes at the wound bed. See, e.g., Nanney L B. Epidermal and dermal effects of epidermal growth factor during wound repair. J Invest Dermatol. 1990; 94:624-9; Brown G L, Curtsinger L J, White M, Mitchell R O, Pietsch J, Nordquist R, et al. Acceleration of tensile strength of incisions treated with EGF and TGF-beta. Ann Surg. 1988; 208:788-94; Nanney L B, Magid M, Stoscheck C M, King L E Jr. Comparison of epidermal growth factor binding and receptor distribution in normal human epidermis and epidermal appendages. J Invest Dermatol. 1984; 83:385-93; Brown G L, Nanney L B, Griffen J, Cramer A B, Yancey J M, Curtsinger L J 3rd, et al. Enhancement of wound healing by topical treatment with epidermal growth factor. N Engl J Med. 1989; 321:76-9; Tsang M W, Wong W K, Hung C S, Lai K M, Tang W, Cheung E Y, et al. Human epidermal growth factor enhances healing of diabetic foot ulcers. Diabetes Care. 2003; 26:1856-61; Viswanathan V. A phase III study to evaluate the safety and efficacy of recombinant human epidermal growth factor (REGEN-D™ 150) in healing diabetic foot ulcers. Wounds. 2006; 18:186-96; Femandez-Montequin J I, Valenzuela-Silva C M, Diaz O G, Savigne W, Sancho-Soutelo N, Rivero-Femandez F, et al. Intra-lesional injections of recombinant human epidermal growth factor promote granulation and healing in advanced diabetic foot ulcers: multicenter, randomised, placebo-controlled, double-blind study. Int Wound J. 2009; 6:432-43.

Recombinant human EGFs including Heberprot-P®, Regen-D™ 150, and Easyef® are commercially available. The present invention contemplates that these EGF molecules may be used in produce EGF conjugates.

During the natural wound healing process, platelets are one of the first cell types to respond at or around the wound site, and pivotal to generating and initiating wound healing.

Platelets have been used as a rich source of growth factors including PDGF. PDGF is produced by platelets, macrophages, endothelial cells, fibroblasts, and keratinocytes. PDGF has been found to regulate cell growth and division and play a role in angiogenesis. It is a potent mitogen and chemoattractant for mesenchymal cells.

PDGF is the first and only recombinant growth factor approved by the Food and Drug Administration (FDA) in the USA for topical administration and is used for the treatment of diabetic foot ulcers. In a randomized controlled trial (RCT), a topical gel containing PDGF-BB (Regranex®) was compared with a placebo in 118 patients with non-healing diabetic ulcers enrolled from 10 different centers. Patients were treated for 20 weeks or until complete wound closure. Of the patients treated with PDGF, 48% healed compared with 25% of the patients treated with the placebo. Topical applications of PDGF to pressure ulcers and venous ulcers have been attempted with minimal efficacy. See, e.g., Tabata Y. Nanomaterials of drug delivery systems for tissue regeneration. Methods Mol Biol. 2005; 300:81-100; Kiwanuka E, Junker J, Eriksson E. Harnessing growth factors to influence wound healing. Clin Plast Surg. 2012; 39:239-48; Salgado A J, Coutinho O P, Reis R L. Bone tissue engineering: state of the art and future trends. Macromol Biosci. 2004; 4:743-65; Schilephake H. Bone growth factors in maxillofacial skeletal reconstruction. Int J Oral Maxillofac Surg. 2002; 31:469-84; Canalis E, McCarthy T L, Centrella M. Effects of platelet-derived growth factor on bone formation in vitro. J Cell Physiol. 1989; 140:530-7; Steed D L. Clinical evaluation of recombinant human platelet-derived growth factor for the treatment of lower extremity ulcers. Plast Reconstr Surg. 2006; 117:143S-9S discussion 50S-51S; Papanas N, Maltezos E. Benefit-risk assessment of becaplermin in the treatment of diabetic foot ulcers. Drug Saf. 2010; 33:455-61; Rees R S, Robson M C, Smiell J M, Perry B H. Becaplermin gel in the treatment of pressure ulcers: a phase II randomized, double-blind, placebo-controlled study. Wound Repair Regen. 1999; 7:141-7; Margolis D J, Morris L M, Papadopoulos M, Weinberg L, Filip J C, Lang S A, et al. Phase I study of H5.020CMV.PDGF-beta to treat venous leg ulcer disease. Mol Ther. 2009; 17:1822-9.

The present invention contemplates that PDGF conjugates as described herein will have superior effects in wound healing.

The VEGF family is composed of VEGF-A, VEGF-B, VEGF-C, VEGF-D, VEGF-E, and placental growth factor. Within this subset of proteins, VEGF-A is the best studied and has a notable role in initiating angiogenesis through the proliferation and migration of endothelial cells. VEGF-A is secreted by platelets and macrophages in response to tissue injury in early wound healing. In addition, hypoxia secondary to metabolic dysfunction is a major stimulus for the release of VEGF-A into the wound microenvironment. Another clinical study shows that VEGF-A improves re-epithelialization of diabetic foot wounds associated with enhanced vessel formation. VEGF165, a recombinant human-VEGF (rh-VEGF) gene carrying plasmid, has been used in only patients with diabetic and ischemic wounds. Randomized controlled trials have been conducted on the efficacy of topical application of rh-VEGF in patients with neuropathic diabetic foot ulcers. In the study, there were positive trends suggestive of potential signals of biological activity observed for incidence of ulcer healing. Compared with other growth factors, relatively few attempts have been made to use VEGF as an adjunctive treatment in wound healing. See, e.g., Barrientos S, Stojadinovic O, Golinko M S, Brem H, Tomic-Canic M. Growth factors and cytokines in wound healing. Wound Repair Regen. 2008; 16:585-601; Senger D R, Ledbetter S R, Claffey K P, Papadopoulos-Sergiou A, Peruzzi C A, Detmar M. Stimulation of endothelial cell migration by vascular permeability factor/vascular endothelial growth factor through cooperative mechanisms involving the alphavbeta3 integrin, osteopontin, and thrombin. Am J Pathol. 1996; 149:293-305; Lokmic Z, Musyoka J, Hewitson T D, Darby I A. Hypoxia and hypoxia signaling in tissue repair and fibrosis. Int Rev Cell Mol Biol. 2012; 296:139-85; Galiano R D, Tepper O M, Pelo C R, Bhatt K A, Callaghan M, Bastidas N, et al. Topical vascular endothelial growth factor accelerates diabetic wound healing through increased angiogenesis and by mobilizing and recruiting bone marrow-derived cells. Am J Pathol. 2004; 164:1935-47; Hanft J R, Pollak R A, Barbul A, van Gils C, Kwon P S, Gray S M, et al. Phase I trial on the safety of topical rhVEGF on chronic neuropathic diabetic foot ulcers. J Wound Care. 2008; 17:30-2 4-7; Baumgartner I, Pieczek A, Manor O, Blair R, Kearney M, Walsh K, et al. Constitutive expression of phVEGF165 after intramuscular gene transfer promotes collateral vessel development in patients with critical limb ischemia. Circulation. 1998; 97:1114-23; Mineur P, Colige A C, Deroanne C F, Dubail J, Kesteloot F, Habraken Y, et al. Newly identified biologically active and proteolysis-resistant VEGF-A isoform VEGF111 is induced by genotoxic agents. J Cell Biol. 2007; 179:1261-73; Chen H X, Cleck J N. Adverse effects of anticancer agents that target the VEGF pathway. Nat Rev Clin Oncol. 2009; 6:465-77.

The present invention contemplates that VEGF conjugates as described herein will have superior effects in wound healing.

The FGF family comprises over 20 isoforms known for their unique mechanism of action that involves binding to proteoglycans in the extracellular matrix (ECM). In general, the FGF proteins are potent mitogens that are instrumental in both normal growth and the wound healing process. Of these proteins, FGF-2, known as bFGF, is the best studied and has a confirmed role in the proliferation of both epithelial and mesenchymal cells as well as a possible role in angiogenesis. See, e.g., Mohammadi M, Olsen S K, Ibrahimi O A. Structural basis for fibroblast growth factor receptor activation. Cytokine Growth Factor Rev. 2005; 16:107-37; Nakamizo S, Egawa G, Doi H, Natsuaki Y, Miyachi Y, Kabashima K. Topical treatment with basic fibroblast growth factor promotes wound healing and barrier recovery induced by skin abrasion. Skin Pharmacol Physiol. 2013; 26:22-9; Robson M C, Hill D P, Smith P D, Wang X, Meyer-Siegler K, Ko F, et al. Sequential cytokine therapy for pressure ulcers: clinical and mechanistic response. Ann Surg. 2000; 231:600-11; Ohura T, Nakajo T, Moriguchi T, Oka H, Tachi M, Ohura N Jr, et al. Clinical efficacy of basic fibroblast growth factor on pressure ulcers: case-control pairing study using a new evaluation method. Wound Repair Regen. 2011; 19:542-51; Richard J L, Parer-Richard C, Daures J P, Clouet S, Vannereau D, Bringer J, et al. Effect of topical basic fibroblast growth factor on the healing of chronic diabetic neuropathic ulcer of the foot. A pilot, randomized, double-blind, placebo-controlled study. Diabetes Care. 1995; 18:64-9; Robson M C, Phillips T J, Falanga V, Odenheimer D J, Parish L C, Jensen J L, et al. Randomized trial of topically applied repifermin (recombinant human keratinocyte growth factor-2) to accelerate wound healing in venous ulcers. Wound Repair Regen. 2001; 9:347-52; Fu X, Shen Z, Chen Y, Xie J, Guo Z, Zhang M, et al. Randomised placebo-controlled trial of use of topical recombinant bovine basic fibroblast growth factor for second-degree burns. Lancet. 1998; 352:1661-4; Hayashida K, Akita S. Quality of pediatric second-degree burn wound scars following the application of basic fibroblast growth factor: results of a randomized, controlled pilot study. Ostomy Wound Manage. 2012; 58:32-6; Hayashida K, Akita S. Surgical treatment algorithms for post-burn contractures. Burns & trauma. 2017; 5:9; Akita S, Akino K, Yakabe A, Tanaka K, Anraku K, Yano H, et al. Basic fibroblast growth factor is beneficial for postoperative color uniformity in split-thickness skin grafting. Wound Repair Regen. 2010; 18:560-6.

Robson et al. treated 61 pressure ulcers with bFGF, GM-CSF, or placebo. Ulcers treated with bFGF alone demonstrated the best healing with regard to wound closure and had elevated levels of bFGF, PDGF, and TGF-β1 in the wound fluid. Similar findings were reported by Ohura et al., where treatment of pressure ulcers with exogenous bFGF resulted in accelerated healing. Administration of bFGF to diabetic foot ulcers provided no significant effects on healing. FGF-10 has been successful in improving the healing rate of non-healing venous ulcers, albeit less extensively tested.

bFGF has also been used as an adjunctive treatment for burn wounds and fractures. Fu et al. did a prospective randomized double-blind multicenter trial to assess the effect of topical recombinant bFGF on burns. They recruited 600 patients and described that the use of bFGF accelerated wound healing. Since burn wounds could be closed rapidly and the patient's own skin soon became available for harvest and autografting, they concluded this growth factor had clinical benefits. Fiblast® Spray is a commercially available recombinant human bFGF product indicated skin ulcers including leg ulcers and burn ulcers. Hayashida et al. reported that partial-thickness burn wounds in pediatric patients treated with bFGF exhibited accelerated healing, reduced scarring, and improved color matching with normal skin compared with controls up to half a year post-operatively. Akita et al. reported similar results in adult patients with burn wounds.

Other FGF proteins involved in wound healing are FGF-1, FGF-7, and FGF-10. FGF-1 is also known as aFGF. Acidic FGF is another classic and well-characterized member of the FGF family, and its structure, binding receptors, and biologic functions are similar to those of the bFGF. Ma et al. performed a randomized, multicenter, double-blind, and placebo-controlled clinical trial to assess the effect of topical aFGF on the healing of skin wounds. In the study, 39 patients with deep-partial burns were included. The assessment results showed that the fully healed rate of the aFGF group was higher than that of the placebo group (53.85% vs 71.79%) in deep-partial burn wounds, and the mean healed time of the burn wounds treated by aFGF was significantly shorter than that of the placebo group (17.23±0.53 vs 18.92±0.49, p=0.035). The results of their clinical trial showed that the wound healing process was faster and the healing time was also shortened in the aFGF-treated group. See, e.g., Ma B, Cheng D S, Xia Z F, Ben D F, Lu W, Cao Z F, et al. Randomized, multicenter, double-blind, and placebo-controlled trial using topical recombinant human acidic fibroblast growth factor for deep partial-thickness burns and skin graft donor site. Wound Repair Regen. 2007; 15:795-9.

FGF-7, also known as KGF or palifermin, is an FGF protein. It preferentially affects epithelial cells and recruits fibroblasts in order to accelerate granulation tissue formation. Staiano-Coico et al. and Danilenko et al. reported that KGF increased the rate of re-epithelialization and epidermal thickness in full- and partial-thickness wounds on porcine epidermis. FGF binding protein (FGF-BP), originally reported to bind and activate FGF-1 and FGF-2, also interacts with KGF and enhances the activity of low growth factor concentrations. Furthermore, expression of FGF-BP is increased following injury to murine skin, particularly in keratinocytes. Thus, upregulation of FGF-BP following cutaneous injury may promote epithelial repair by stabilizing KGF and possibly providing protection from proteases in the wound environment. See, e.g., Staiano-Coico L, Krueger J G, Rubin J S, D'Limi S, Vallat V P, Valentino L, et al. Human keratinocyte growth factor effects in a porcine model of epidermal wound healing. J Exp Med. 1993; 178:865-78; Danilenko D M, Ring B D, Tarpley J E, Morris B, Van G Y, Morawiecki A, et al. Growth factors in porcine full and partial thickness burn repair. Differing targets and effects of keratinocyte growth factor, platelet-derived growth factor-BB, epidermal growth factor, and neu differentiation factor. Am J Pathol. 1995; 147:1261-77; Beer H D, Bittner M, Niklaus G, Munding C, Max N, Goppelt A, et al. The fibroblast growth factor binding protein is a novel interaction partner of FGF-7, FGF-10 and FGF-22 and regulates FGF activity: implications for epithelial repair. Oncogene. 2005; 24:5269-77. Finch P W, Mark Cross L J, McAuley D F, Farrell C L. Palifermin for the protection and regeneration of epithelial tissues following injury: new findings in basic research and pre-clinical models. J Cell Mol Med. 2013; 17:1065-87.

The present invention contemplates that FGF conjugates as described herein, and including conjugates of bFGF, aFGF, KGF and other FGF-family members will have superior effects in wound healing.

The TGF-β proteins are members of the TGF-β superfamily and exist as three functional isoforms: TGF-β1, TGF-β2, and TGF-β3. In the event of tissue injury, TGF-β is released into the wound microenvironment from storage sites in the ECM and secreted by macrophages, fibroblasts, and platelets. In the early stages of wound healing, TGF-β has a reported role in modulating re-epithelialization, chemotaxis of leukocytes, and angiogenesis. However, the hallmark of TGF-β proteins is their ability to modulate wound contraction and scarring. TGF-β1, 2, and 3 isoforms show a unique expression pattern spatially and temporally during cutaneous wound repair. Though TGF-β1 and TGF-β3 are largely homologous, they may exert opposing effects. In particular, one study suggested that, in contrast to TGF-β1, TGF-β3 may have an anti-fibrotic effect during wound healing and in different tissues: skin and mucosa. Among the three isoforms, TGF-β1 is considered the most important in the process of wound healing. TGF-β1-deficient mice develop massive inflammation, explaining why TGF-β1 has gained the attribute as an anti-inflammatory cytokine. Administration of TGF-β3 (Avotermin; Renovo, UK) significantly reduced scarring in a number of clinical trials before the drug failed to meet its endpoints in stage III clinical trials. See, e.g., Puolakkainen P A, Reed M J, Gombotz W R, Twardzik D R, Abrass I B, Sage H E. Acceleration of wound healing in aged rats by topical application of transforming growth factor-beta (1). Wound Repair Regen. 1995; 3:330-9; Werner S, Grose R. Regulation of wound healing by growth factors and cytokines. Physiol Rev. 2003; 83:835-70; Le M, Naridze R, Morrison J, Biggs L C, Rhea L, Schutte B C, et al. Transforming growth factor beta 3 is required for excisional wound repair in vivo. PLoS One. 2012; 7:e48040; Lichtman M K, Otero-Vinas M, Falanga V. Transforming growth factor beta (TGF-beta) isoforms in wound healing and fibrosis. Wound Repair Regen. 2016; 24:215-22; Chang Z, Kishimoto Y, Hasan A, Welham N V. TGF-beta3 modulates the inflammatory environment and reduces scar formation following vocal fold mucosal injury in rats. Dis Model Mech. 2014; 7:83-91; Kiritsi D, Nystrom A. The role of TGFbeta in wound healing pathologies. Mech Ageing Dev. 2018; 172:51-8; Li M O, Wan Y Y, Sanjabi S, Robertson A K, Flavell R A. Transforming growth factor-beta regulation of immune responses. Annu Rev Immunol. 2006; 24:99-146; Finnson K W, Arany P R, Philip A. Transforming growth factor beta signaling in cutaneous wound healing: lessons learned from animal studies. Advances in wound care. 2013; 2:225-37; Abramov Y, Hirsch E, Ilievski V, Goldberg R P, Botros S M, Sand P K. Transforming growth factor beta 1 gene expression during vaginal vs cutaneous surgical wound expression during vaginal vs cutaneous surgical wound healing in the rabbit. Int Urogynecol J. 2013; 24:671-5; Reid R R, Roy N, Mogford J E, Zimmerman H, Lee C, Mustoe T A. Reduction of hypertrophic scar via retroviral delivery of a dominant negative TGF-beta receptor II. J Plast Reconstr Aesthet Surg. 2007; 60:64-72; Singer A J, Huang S S, Huang J S, McClain S A, Romanov A, Rooney J, et al. A novel TGF-beta antagonist speeds reepithelialization and reduces scarring of partial thickness porcine burns. J Burn Care Res. 2009; 30:329-34.

The present invention contemplates that TGF-βconjugates as described herein, and including conjugates of TGF-β1, TGF-β2, and TGF-β3, will have superior effects in wound healing.

GM-CSF is a cytokine found in the wound bed after acute injury that enables faster wound healing. Specifically, GM-CSF recruits Langerhans cells, stimulates local recruitment of inflammatory cells, advances myofibroblast differentiation to facilitate wound contraction, and mediates proliferation of the epidermis. Several studies involving topical application of GM-CSF have been reported. See, e.g., Barrientos S, Brem I-I, Stojadinovic O, Tomic-Canic M. Clinical application of growth factors and cytokines in wound healing. Wound Repair Regen. 2014; 22:569-78: Jaschke E, Zabernigg A, Gattringer C. Recombinant human granulocyte-macrophage colony-stimulating factor applied locally in low doses enhances healing and prevents recurrence of chronic venous ulcers. Int J Dermatol. 1999; 38:380-6; da Costa R M, Aniceto C, Jesus F M, Mendes M. Quick healing of leg ulcers after molgramostim. Lancet. 1994; 344:481-2; Khan M N, Davies C G. Advances in the management of leg ulcers—the potential role of growth factors. Int Wound J. 2006; 3:113-20; Mayer W, Jochmann W, Partsch H. Varicose ulcer: healing in conservative therapy. A prospective study. Wiener medizinische Wochenschrift (1946). 1994; 144:250-2.

The present invention contemplates that GM-CSF conjugates as described herein will have superior effects in wound healing.

The growth factor conjugates of the present invention may utilize wild-type growth factor molecules (i.e., growth factor molecules having an amino acid sequence found naturally in a species of interest such as a human) or variant growth factors molecules which have one or more mutations or additional amino acids as compared to a wild-type growth factor molecule. In some embodiments, as described in more detail below, the variant growth factor molecules may comprise a tag or linker sequence located at the amino or carboxyl terminal of the growth factor molecule of may contain amino acid substitutions, deletions and/or additions within the growth factor molecule to create a tag site. In other embodiments, the variant growth factor molecule may be a truncated version of the wild-type molecule and contain specific functional domains.

In some preferred embodiments, the chemical moiety R₁ is covalently attached to the growth factor molecule to provide the reactive functionality R₂. A variety of chemical moieties, such as homobifunctional and heterobifunctional cross-linkers may be utilized to provide the growth factor conjugates. Conjugation is the process of chemically modifying a protein molecule by covalent bonding. There are multiple chemical groups in proteins that may be used for conjugation.

Covalent modification and crosslinking of proteins depends on the availability of particular chemicals that are capable of reacting with the specific kinds of functional groups that exist in proteins. In addition, protein function and structure are either the direct focus of study or they must be preserved if a modified protein is to be useful in a technique. Therefore, the composition and structure of proteins, and the potential effects of modification reagents on protein structure and function, must be considered.

Proteins have four levels of structure. The sequence of its amino acids is the primary structure. This sequence is always written from the amino end (N-terminus) to the carboxyl end (C-terminus). Protein secondary structure refers to common repeating elements present in proteins. There are two basic components of secondary structure: the alpha helix and the beta-pleated sheet. Alpha helices are tight, corkscrew-shaped structures formed by single polypeptide chains. Beta-pleated sheets are either parallel or anti-parallel arrangements of polypeptide strands stabilized by hydrogen bonds between adjacent —NH and —CO groups. Parallel beta-sheets have adjacent strands that run in the same direction (i.e., N-termini next to each other), while anti-parallel beta sheets have adjacent strands that run in opposite directions (i.e., N-terminus of one strand arranged toward the C-terminus of adjacent strand). A beta-pleated sheet may contain two to five parallel or antiparallel strands.

Tertiary structure is the full three-dimensional, folded structure of the polypeptide chain and is dependent on the suite of spontaneous and thermodynamically stable interactions between the amino acid side chains. Disulfide bond patterns, as well as ionic and hydrophobic interactions greatly impact tertiary structure. Quaternary structure refers to the spatial arrangement of two or more polypeptide chains. This structure may be a monomer, dimer, trimer, etc. The polypeptide chains composing the quaternary structure of a protein may be identical (e.g., homodimer) or different (e.g., heterodimer).

The complete structure of a functioning protein involves more than polypeptide chains at the four levels of structure. Various covalent modifications often occur, either during or after assembly of the polypeptide chain. Most proteins undergo co- and/or post-translational modifications. Examples include phosphorylation (of serine, threonine or tyrosine residues), glycosylation, and ubiquitination.

Knowledge of native modifications is important because they may alter physical and chemical properties, folding, conformation distribution, stability, activity, and consequently, function of the proteins. Proteins are relatively easy molecules to manipulate, and protein crosslinking and chemical modification methods are commonly used to determine the roles of individual amino acid side chains in the physical, chemical, and biological properties of proteins.

Despite the complexity of protein structure, including composition with 20 different amino acids, only a small number of protein functional groups comprise selectable targets for practical bioconjugation methods. Four protein chemical targets account for the vast majority of crosslinking and chemical modification techniques:

Primary amines (—NH2): This group exists at the N-terminus of each polypeptide chain and in the side chain of lysine (Lys, K) residues.

Carboxyls (—COOH): This group exists at the C-terminus of each polypeptide chain and in the side chains of aspartic acid (Asp, D) and glutamic acid (Glu, E).

Sulfhydryls (—SH): This group exists in the side chain of cysteine (Cys, C). Often, as part of a protein's secondary or tertiary structure, cysteines are joined together between their side chains via disulfide bonds (—S—S—).

Carbonyls (—CHO): These aldehyde groups can be created by oxidizing carbohydrate groups in glycoproteins.

For each of these protein functional-group targets, there exist one to several types of reactive groups that are capable of targeting them, and these have been used as the basis for synthesizing crosslinking and modification reagents. Homobifunctional reagents comprise identical reactive functionalities on either end of a spacer arm. Heterobifunctional reagents comprise different reactive functionalities on either end of a spacer arm. Both types of cross linkers mat be used to make the growth factor conjugated of the present invention.

For example, in some preferred embodiments, a heterobifunctional cross linker is utilized wherein one reactive functionality in the crosslinker covalently binds to a primary amine, carboxyl, sulfhydryl or carbonyl group in the growth factor molecule and other reactive functional group reacts with a different functional group in the wound bed. In some preferred embodiments, the crosslinker comprises a first reactive functionality that reacts with an amine, carboxyl, or carbonyl group in the growth factor. The second reactive functionality preferably reacts with a sulfhydryl group, such as would be present in a wound bed. Accordingly, in some preferred embodiments, covalent modification of the growth factor with a heterobifunctional crosslinker thus provides a growth factor conjugate of the present invention which presents a reactive functionality (in this case a sulfhydryl reactive functionality) so that the conjugate can be covalently attached to the wound bed. In some preferred embodiments, the first reactive functionality is a N-hydroxysuccinimide (NHS) ester group (which reacts with primary amines) and the second reactive functionality is a maleimide, bromoacetyl, or iodoacetyl group (which reacts with sulfhydryls). In some preferred embodiments, the cross linker is succinimidyl 4-(N maleimidomethyl)cyclohexane-1-carboxylate (SMCC), most preferably sulfo-SMCC. Other suitable cross linkers include: N-α-maleimidoacet-oxysuccinimide ester (AMAS), N-β-maleimidopropyl-oxysuccinimide ester (BMPS), N-ε-maleimidocaproic acid (EMCA), N-ε-malemidocaproyl-oxysuccinimide ester (EMCS), N-γ-maleimidobutyryl-oxysuccinimide ester (GMBS), succinimidyl 4-(N-maleimidomethyl)cyclohexane-1-carboxy-(6-amidocaproate) (LC-SMCC), succinimidyl 6-(3(2-pyridyldithio)propionamido)hexanoate (LC-SPDP), m-maleimidobenzoyl-N-hydroxysuccinimide ester (MBS), pegylated-SPDP, succinimidyl 3-(bromoacetamido)propionate (SBAP), succinimidyl iodoacetate (SIA), succinimidyl (4-iodoacetyl)aminobenzoate (SIAB), SM(PEG)2-12, succinimidyl 4-(p-maleimidophenyl)butyrate (SMBP), Succinimidyl 6-((beta-maleimidopropionamido)hexanoate)(SMPH), 4-succinimidyloxycarbonyl-alpha-methyl-α(2-pyridyldithio)toluene (SMPT), SPDP, Sulfo-ECMS, Sulfo-GMBS, Sulfo-KMUS, Sulfo-LC-SPDP, Sulfo-MBS, Sulfo-SIAB, and Sulfo-SMBP.

The present invention is not limited to the use of any particular reactive functionalities or crosslinkers. For example, in other preferred embodiments, the first reactive functionality is a hydrazide group (which reacts with primary amines) and the second reactive functionality is a maleimide group or pyridyldisulfide group (which reacts with sulfhydryls). Example of suitable crosslinkers include: N-β-maleimidopropionic acid hydrazide (BMPH), N-ε-maleimidocaproic acid hydrazide (EMCH), N-κ-maleimidoundecanoic acid hydrazide (KMUH), 4-(4-N-maleimidophenyl)butyric acid hydrazide (MPBH), and 3-(2-pyridyldithio)propionyl hydrazide (PDPH).

As discussed above, in some preferred embodiments, the growth factor may be a variant growth factor. In some preferred embodiments, the growth factor is engineered to comprise an aldehyde tag sequence at either the amino terminus (N-terminal) or carboxy terminus (C-terminal) of the growth factor or within the growth factor molecule itself. Suitable aldehyde tags include LCTPSRAALLTGR (ALD₁₃: SEQ ID NO:1) and LCTPSR (ALD₆; SEQ ID NO:2). By “aldehyde tag” or “ald-tag” is meant an amino acid sequence that contains an amino acid sequence derived from a sulfatase motif which is capable of being converted, or which has been converted, by action of a formylglycine generating enzyme (FGE) to contain a 2-formylglycine residue (referred to herein as “fGly”). The fGly residue generated by an FGE is often referred to in the literature as a “formylglycine”. Stated differently, the term “aldehyde tag” is used herein to refer to an amino acid sequence comprising an “unconverted” sulfatase motif (i.e., a sulfatase motif in which the cysteine or serine residues has not been converted to fGly by an FGE, but is capable of being converted, such as a sulfatase motif with the sequence: L(C/S)TPSR (SEQ ID NO:3)) as well as to an amino acid sequence comprising a “converted” sulfatase motif (i.e., a sulfatase motif in which the cysteine or the serine residue has been converted to fGly by action of an FGE, e.g., L(fGly)TPSR (SEQ ID NO:4)). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 9,579,390, the disclosure of each of which is incorporated herein by reference in its entirety. In these embodiments, the crosslinker preferably comprises at least one group that is reactive with the fGly residue and can preferably be a hydrazide group as described above. The other reactive group can preferably be a maleimide, bromoacetyl, or iodoacetyl group as described above.

Other homobifunctional crosslinkers that can be used to make conjugates of the present invention include, but are not limited to, N-hydroxysuccinimidyl ester (e.g., including, but not limited to, disuccinimidyl ester, dithiobis(succinimidylpropionate), 3,3′-dithiobis(sulfosuccinimidylpropionate), disuccinimidyl suberate, bis(sulfosuccinimidyl)suberate, disuccinimidyl tartarate, disulfosuccinimidyl tartarate, bis[2-(succinimidyloxycarbonyloxy)ethyl]sulfone, bis[2-(sulfosuccinimidooxycarbonyloxy)ethyl]sulfone, ethylene glycolbis(succinimidylsuccinate), ethylene glycolbis(sulfosuccinimidylsuccinate), disuccinimidyl glutarate, and N,N′-disuccinimidylcarbonate).

Accordingly, in preferred embodiments, the present invention provides growth factor conjugates comprising a growth factor to which a chemical moiety displaying a reactive functionality is attached. The reactive functionality is preferably reactive with chemical groups, invention provides methods of treating wounds, such as surgical wounds, burn wounds, and chronic wounds. In particularly preferred embodiments, the wounds are chronic wounds.

The present invention is not limited to the treatment of any particular type of chronic wounds. Indeed, the treatment of a variety of chronic wounds is contemplated, including but not limited to the following types of chronic wounds. Diabetic ulcers are wounds that occur on the foot, heel or toes of diabetics. Often there is little or no feeling in the feet or in the ulcer itself. In some embodiments, the diabetic ulcer is a diabetic foot ulcer. Pressure ulcers (also known as bedsores are caused by prolonged sitting or lying in one position long enough to damage the skin. The ulcer may be very painful and may drain a little or a large amount. They commonly develop on the tailbone, heels, elbows, shoulder blades, knees, ankles, and the back of the head or the spine. Traumatic ulcers occur when trauma or injury to the body may cause a loss of tissue layers and may result in damage to or loss of body parts or organs. Trauma may also compromise the arterial, venous, or lymphatic systems of the body causing these common wounds. Arterial ulcers are round-shaped wounds caused by impaired circulation and is often seen on the legs or feet. Venous stasis ulcers occur when blood pools in the veins. This pooling causes swelling and an ulcer develops. A venous stasis ulcer drains heavily, is irregular in shape and is often painful. Venous stasis ulcers commonly occur in the lower leg between the ankle and the knee. Post-surgical wounds are a common complication of surgery. Signs of these common wounds including redness around a surgical wound in the days following surgery as well as redness or streaking in surrounding tissues, throbbing or intense pain, pus or collection of fluids around the surgical site, a strong, unpleasant odor, and a mild to moderate fever.

In some preferred embodiments, one or more growth factor conjugates of the present invention are applied to the wound under conditions such that growth factor conjugates covalently bind to chemical groups in the wound bed. The growth factor conjugates of the present invention may be used alone, for example an EGF conjugate, or may be combined, for example an EGF conjugate and PDGF conjugate. The growth factors conjugates are preferably administered in an aqueous solution, although other delivery forms commonly used for topical administration may be utilized including gels, hydrogels, creams, ointments, sprays, powders and the like.

In some embodiments, the conjugates are provided in a polymeric delivery vehicle such as a polymeric matrix. In some embodiments, the polymeric delivery vehicle is a matrix formed from a biocompatible polymer, for example, polyvinyl alcohol (PVA), polyacrylic acid (PAA), polystyrene (PS), PMMA polymethyl methacrylate (PMMA), or polyvinylacetate (PVAc). In some preferred embodiments, the polymeric matrix is water soluble.

In some preferred embodiments, polymeric delivery vehicle is a polyelectrolyte membrane (PEM). Suitable PEMs are described in U.S. application Ser. No. 14/440,997, which is incorporated herein by reference in its entirety. Polyelectrolyte layers are formed by alternating applications of anionic polyelectrolytes and cationic polyelectrolytes to surfaces to form a polyelectrolyte multilayer. In some embodiments, one or more wound active agents, such as those described above, are incorporated into the multilayer. Preferably, at least four layers, and, more preferably, at least six layers are used to form the polyelectrolyte multilayer.

Cationic polyelectrolytes useful in the present invention can be any biocompatible water-soluble polycationic polymer, for example, any polymer having protonated heterocycles attached as pendant groups. As used herein, “water soluble” means that the entire polymer must be soluble in aqueous solutions, such as buffered saline or buffered saline with small amounts of added organic solvents as co-solvents, at a temperature between 20 and 37° Centigrade. In some embodiments, the material will not be sufficiently soluble (defined herein as soluble to the extent of at least one gram per liter) in aqueous solutions per se but can be brought into solution by grafting the polycationic polymer with water-soluble polynonionic materials such as polyethylene glycol.

Representative cationic polyelectrolytes include natural and unnatural polyamino acids having net positive charge at neutral pH, positively charged polysaccharides, and positively charged synthetic polymers. Examples of suitable polycationic materials include i5 polyamines having amine groups on either the polymer backbone or the polymer side chains, such as poly-L-lysine (PLL) and other positively charged polyamino acids of natural or synthetic amino acids or mixtures of amino acids, including, but not limited to, poly(D-lysine), poly(omithine), poly(arginine), and poly(histidine), and nonpeptide polyamines such as poly(aminostyrene), poly(aminoacrylate), poly (N-methyl aminoacrylate), poly (N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), and natural or synthetic polysaccharides such as chitosan.

In general, the polymers must include at least five charges, and the molecular weight of the polycationic material must be sufficient to yield the desired degree of binding to a tissue or other surface, having a molecular weight of at least 1000 g/mole.

Polyanionic materials useful in the present invention can be any biocompatible water-soluble polyanionic polymer, for example, any polymer having carboxylic acid groups attached as pendant groups. Suitable materials include alginate, carrageenan, furcellaran, pectin, xanthan, hyaluronic acid, heparin, heparan sulfate, chondroitin sulfate, polyacrylic acid (PAA), dermatan sulfate, dextran sulfate, poly(meth)acrylic acid, oxidized cellulose, carboxymethyl cellulose and crosmarmelose, synthetic polymers and copolymers containing pendant carboxyl groups, such as those containing maleic acid or fumaric acid in the backbone. Polyaminoacids of predominantly negative charge are also suitable. Examples of these materials include polyaspartic acid, polyglutamic acid, and copolymers thereof with other natural and unnatural amino acids. Polyphenolic materials such as tannins and lignins can be used if they are sufficiently biocompatible. Preferred materials include alginate, pectin, carboxymethyl cellulose, heparin and hyaluronic acid.

In some embodiments, the cationic polyelectrolyte used is PLL and the anionic polyelectrolyte used is poly(L-glutamic acid) (PGA). Indeed, the use of a variety of polyelectrolytes is contemplated, including, but not limited to, poly(ethylene imine) (PEI), poly(allylamine hydrochloride) (PAH), poly(sodium 4-styrenesulfonate) (PSS), poly(acrylic acid) (PAC), poly(maleic acid-co-propylene) (PMA-P), and poly(vinyl sulfate) (PVS). It is also possible to use naturally occurring polyelectrolytes, including hyaluronic acid and chondroitin sulfate. In still further embodiments, the polymer is a dendrimer, grafted polymer, or star architecture polymer.

In some embodiments, the multilayer structures are formed from uncharged polymers or from a combination of charged and uncharged polymers. Examples of uncharged polymers include, but are not limited to, dextran, dextran sulfate, diethylaminoethyl (DEAE)-dextran, hydroxyethyl cellulose, ethyl(hydroxyethyl) cellulose, acrylamide, polyethylene oxide, polypropylene oxide, polyethylene oxide—polypropylene oxide copolymers, PAANa, Ficoll, polyvinylpyrolidine, and polyacrylic acid.

In some embodiments, the multilayer structures are formed from one or more amphoteric polymers, alone in combination with the other polymers described herein. In some embodiments, the amphoteric polymers comprise one or more of acrylic acid (AA), DMAEMA (dimethylaminoethyl methacrylate), APA (2-aminopropyl acrylate), MorphEMA (morpholinoethyl methacrylate), DEAEMA (diethylaminoethyl methacrylate), t-ButylAEMA (t-butylaminoethyl methacrylate), PipEMA (piperidinoethyl methacrylate), AEMA (aminoethyl methacrylate), HEMA (2-hydroxyethyl methacrylate), MA (methyl acrylate), MAA (methacrylic acid) APMA (2-aminopropyl methacrylate), AEA (aminoethyl acrylate). In some embodiments, the amphoteric polymer comprises (a) carboxylic acid, (b) primary amine, and (c) secondary and/or tertiary amine. The amphoteric polymers have an isoelectric point of 4 to 8, preferably 5 to 7 and have a number average molecular weight in the range of 10,000 to 150,000. Polymer layers may be formed by a variety of methods. In some embodiments, the polymer layers are formed on solid supports as described in detail below. In some embodiments, it is contemplated that the polymer or polymer multilayer is formed by sequential application of polymers using either a pump (including syringes, ink jet printers, and electrojets) or spray, such as an aerosol spray, or by dip coating. In other embodiments, particle bombardment is utilized. In other embodiments, the use of a brush including an air brush is contemplated. In other embodiments, a sponge is utilized. In other embodiments a solid support or stamp such as an elastomeric material, for example, PDMS (polydimethylsiloxane), silicone, hydrogel or latex, is used to support the polymer layer and mechanically transfer the polymer layer into or onto the wound bed. In still other embodiments, the nanoscale polymer matrix is formed on a solid support, a sacrificial polymer layer is formed on the nanoscale polymer matrix, and the resulting microsheet is peeled from the solid support.

In some embodiments, the wound to be treated has a wound surface. In some preferred embodiments, the conjugate(s) is applied to the wound surface in an amount sufficient to cover the wound surface with from 0.1 to 20 pg of the of the covalently attached conjugate per mm² of the wound surface. In some preferred embodiments, the conjugate(s) is applied to the wound surface in an amount sufficient to cover the wound surface with from 0.1 to 10 pg of the of the covalently attached conjugate per mm² of the wound surface. In some preferred embodiments, the conjugate(s) is applied to the wound surface in an amount sufficient to cover the wound surface with from 0.5 to 5 pg of the of the covalently attached conjugate per mm² of the wound surface. The amount of the conjugate applied to the wound will vary with the size if the wound, but in general from about 1 to 200 nanograms and more preferably from about 1 to 100 nanograms of the conjugate will be applied to the wound per treatment to provide the targeted amount of covalent attachment of the conjugate to the wound surface.

The present invention contemplates that the conjugate may be applied one or more time to the wound surface. In some preferred embodiments, the conjugate is applied every 24 to 72 hours. In some preferred embodiments, the conjugate is applied every 36 to 60 hours. In some preferred embodiments, the conjugate is applied until more than 50% of the wound is re-epithelialized. In some preferred embodiments, the conjugate is applied until more than 75% of the wound is re-epithelialized. In some preferred embodiments, the conjugate is applied until more than 90% of the wound is re-epithelialized. In some preferred embodiments, the conjugate is applied until more than 95% of the wound is re-epithelialized. In some preferred embodiments, the conjugate is applied to the wound from about 3 to about 30 times. In some preferred embodiments, the conjugate is applied to the wound from about 3 to about 20 times. In some preferred embodiments, the conjugate is applied to the wound from about 5 to about 15 times.

### EXPERIMENTAL

### Materials and Methods

Preparation of EGF: (i) Soluble EGF: Human recombinant EGF (ProspecBio) was administered at 300 ng per treatment dissolved in 5% (w/v) polyethylene glycol (PEG) in a total volume of 30 μl. (ii) Crosslinked EGF: EGF was conjugated with sulfosuccinimidyl-4-(N-maleimidomethyl)cyclohexane-1-carboxylate (s-SMCC; ThermoPierce) following manufacturer's protocol. Conjugation efficiency was determined by the well-established TNBS assay (ThermoPierce) to determine reduction in free-amine groups of s-SMCC following manufacturers protocol. (iii) Quenched EGF: s-SMCC conjugated EGF was incubated with 10 molar excess of β-mercaptoethanol (FisherSci) and dialyzed against PBS to remove unreactive β-mercaptoethanol. (iv) Vehicle control: 5% (w/v) PEG in Hank's Balanced Salt Solution was used as vehicle control.

In vitro surface modification: An in vitro model of the wound surface was generated by silanizing glass coverslips with either trimethoxy(propyl)silane (TPS) or 3-mercaptopropyl trimethoxysilane (3-MPTS). Disulfide bonds (if any) on the surface of the silanated coverslips were reduced using 10 μM tris(2-carboxyethyl)phosphine (TCEP) for 10 min to generate an abundance of —SH bonds. These exposed —SH groups were used to covalently immobilize EGF.

Confirming surface covalent immobilization: In order to confirm that —SH reactive groups were able to bind to malemide groups on the crosslinker, we used methoxyPEG (5000 mw) or methoxy PEG-malemide (5000 mw) for these experiments. (i) Goniometry: Unmodified glass coverslips, and TPS or 3-MPTS modified glass coverslips were treated with TCEP, and incubated with methoxyPEG or methoxyPEG-malemide for 10 min, washed thoroughly with HBSS, and air dried. Using a Ramé-Hart goniometer, the contact angle on all surfaces were determined using deionized water. (ii) Ellipsometry: Unmodified silicon wafer (Si/SiO2), and TPS or 3-MPTS modified silicon wafer were treated with TCEP, and incubated with methoxyPEG or methoxyPEG-malemide for 10 min, washed thoroughly with HBSS, and air dried. Using an Elli2000 (NanoFilm technologie, Germany), ellipsometric angles (Δ and ω) were resolved. Measurements were taken at an incidence angle of 60°.

Measuring bioactivity in vitro: Human immortalized kertinocytes (HaCaT cells) were plated on unmodified glass surfaces, or on surfaces covalently immobilized with EGF, or on surfaced with quenched EGF, in Dulbeccos' modified Eagle's medium (DMEM) supplemented with 1% fetal bovine serum and proliferation was determined after 5 days using the MTT assay (Raghunathan et al., 2013). Cells were also cultured in growth medium containing 10% serum for comparison.

In vivo studies: All procedures adhered to the recommendations in the Guide for the Care and Use of Laboratory Animals of the National Institutes of Health. The protocol for this experiment was approved by the Institutional Animal Care and Use Committee of University of California, Davis.

Creating of full thickness splinted dermal wounds: Genetically diabetic 12 week old male mice (db/db; BKS.Cg-m+/+Leprdb; Jackson Laboratories, Bar Harbor, ME) were used for this study. The average 6 SEM body weight of the mice was 42±0.5 g. 8 mm Full thickness wounds were created on the dorsal surface of db/db mice as described previously (Park S. A. et al., 2014).

Treatment of wounds: The wounds were treated with 10 μM TCEP for 10 min following which either covalently modified human recombinant EGF (CxEGF), quenched EGF (qEGF) or native soluble EGF (sEGF) was applied onto the wound bed for 30 min prior to application of the bandages. Mice in the sEGF group received native EGF (300 ng) dissolved in PEG immediately after wounding and twice daily until day 10. The NoTx group remained without any treatment through the study period. All wounds were dressed using a sterile coverslip and a semi-occlusive bandage. Mice in the CxEGF-6x group received TCEP and covalently modified human recombinant EGF (30 ng) once immediately after wounding and once every other day over 11 days.

Clinical and histological analyses: All analyses were performed as described previously (Park S. A. et al., 2014). Briefly, wounds were clinically evaluated and imaged daily, and the wound area was measured using image analysis software, and percentage wound closure was calculated. Mice were humanely euthanized on day 11 and the entire wound bed was harvested. Histologic analyses was performed by a board-certified veterinary pathologist.

Detection of crosslinked EGF on dermal wounds: Persistence of CxEGF, qEGF and soluble EGF in the wound bed was determined using ELISA specific to anti-human EGF. Mice were euthanized at each time point and the wound bed was harvested. The tissue was digested using 7.5 Units/ml collagenase in the presence of 1 μM dithiothreitol (DTT) over 2 h at 37° C. This was centrifuged at 14000 RPM and the supernatant following which ELISA was performed. Specificity of the ELISA was verified using mouse recombinant EGF. Amount of EGF (mg) measured on the wound bed was expressed per surface area (mm²) of the tissue.

### Results & Discussion

### Impact of Topically Applied Growth Factor on Wound Healing

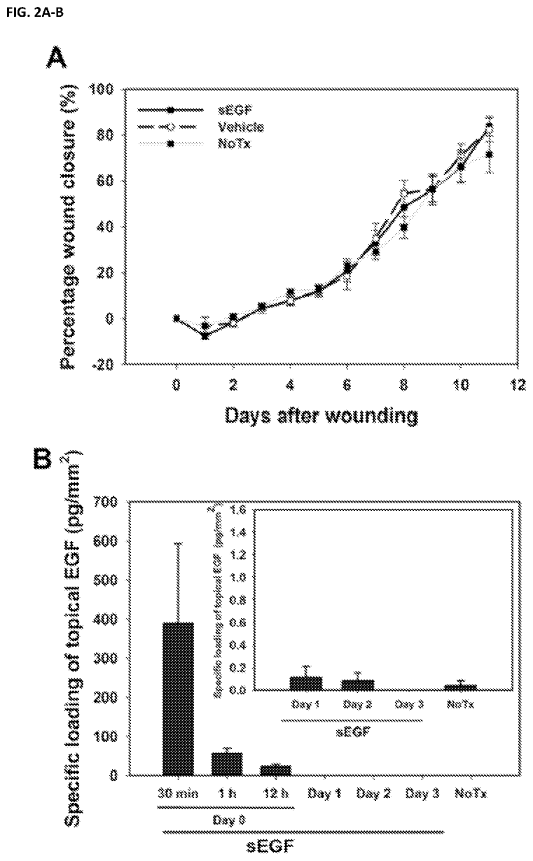

EGF is a key growth factor involved in triggering wound re-epithelialization, one of the critical early events in wound closure, whose prolonged exposure is required for eliciting a cellular response (Bennett N. T. and Schultz G. S., 1993, Bennett N. T. and Schultz G. S., 1993, 171 Gipson and Inatomi, 1995, Jost et al., 2000). We therefore chose to test whether soluble EGF resulted in accelerated re-epithelialization in diabetic mice. A full thickness splinted wound (8 mm in diameter) was created on each heterozygous (db+/−; BKS.Cg-m+/+ Leprdb) mouse as described previously (Park Shin Ae et al., 2014). Soluble EGF (0.001% (v/v) sEGF; 300 ng per 0.5 mm² wound), topically administered twice daily, to full thickness splinted wounds did not significantly alter wound healing or epithelial closure in diabetic mice (db/db; FIG. 2, FIG. 7). Functional bioactivity of EGF was determined in vitro using spontaneously transformed keratinocytes (HaCaT cell line) from histologically normal skin (FIG. 8). As a possible contributor to the lack of significant positive effect, we observed a rapid loss of recombinant human EGF from the surface of wounds within 1 h of topical administration (FIG. 2B). Efficient delivery of cytoactive factor-based therapeutics can be affected by wound exudate, high concentrations of matrix metalloproteinases (MMPs), and absorbent bandages wicking soluble factors away from the wound surface. We hypothesized that the failure of EGF to promote wound healing was directly related to its rapid disappearance from the wound surface and that strategies to increase retention of EGF on wound surface would promote re-epithelialization.

A number of strategies, including bio-functionalized scaffolds, micro-/nano-particles and cytokine gels, have been investigated as adjunctive therapeutics for the acceleration of wound healing and reduction of bioburden (Blaker et al., 2004, Boateng et al., 2013, Pawar et al., 2014, Pawar et al., 2013, Rennekampff et al., 2000, Schneider et al., 2009), though very few are commercially available. Covalent immobilization of cytoactive factors to polymeric/metallic substrates improves retention and bioavailability of molecules to cells and has demonstrable success in in vitro approaches (Chattopadhyay et al., 2014, Jain et al., 2013, Masters, 2011, Stefonek-Puccinelli and Masters, 2008, Stefonek and Masters, 2007). However, these involve complex scaffold fabrication methodologies and subsequent surface modification steps that are time consuming and expensive, and as such have yielded very little gain. More recently, Martino et al. reported engineered growth factors with super-affinity to the native ECM, which yet again involves complex syntheses (Martino et al., 2011). Therefore, we developed a simple delivery method that exploits surface chemistry of the native wound bed for anchoring growth factors. Particularly, we demonstrate that cytoactive factors, such as EGF, can be covalently integrated directly into the wound surface, without the use of any biomaterial carriers, to accelerate re-epithelialization, a new paradigm for the delivery of cytoactive and antimicrobial therapeutics to improve wound healing outcomes.

### In Vitro Validation of Covalently Immobilized EGF

To achieve immobilization of growth factors, we used sulfosuccinimidyl-4-(N-maleimidomethyl)cyclohexane-1-carboxylate (Sulfo-SMCC) as a water soluble heterobifunctional crosslinker with amine reactive NHS ester and a sulhydryl reactive malemide group. The native dermis presents an environment rich in cysteines and disulfides (Jain et al., 210 2013) that can be exploited to immobilize growth factors chemically modified with sulfo-SMCC.

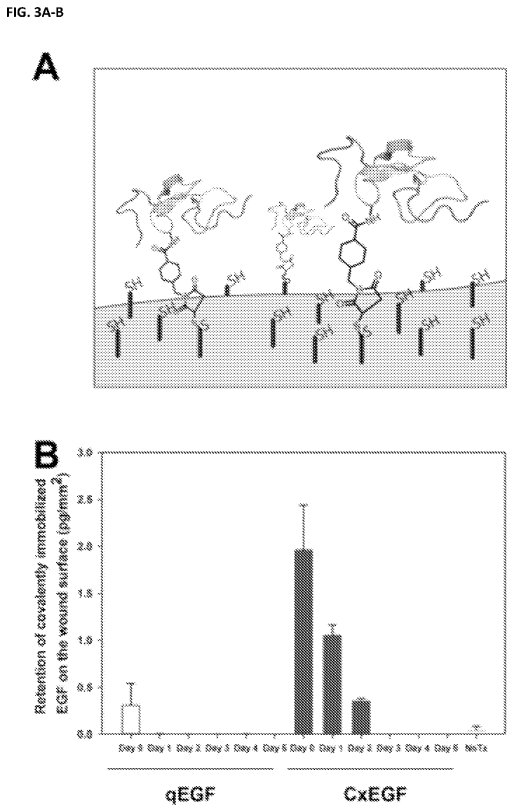

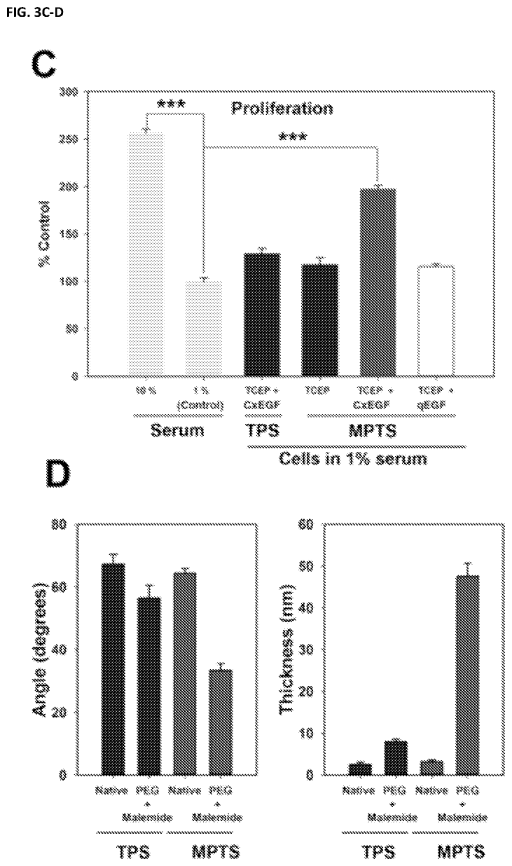

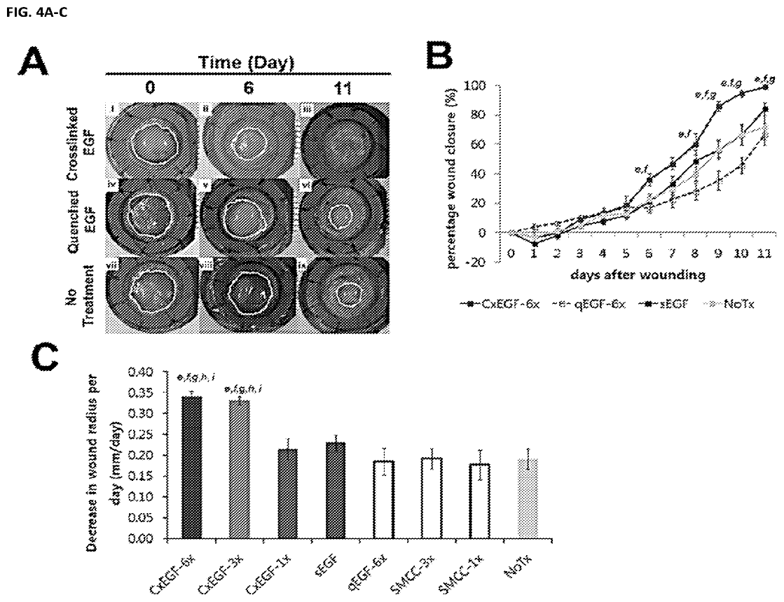

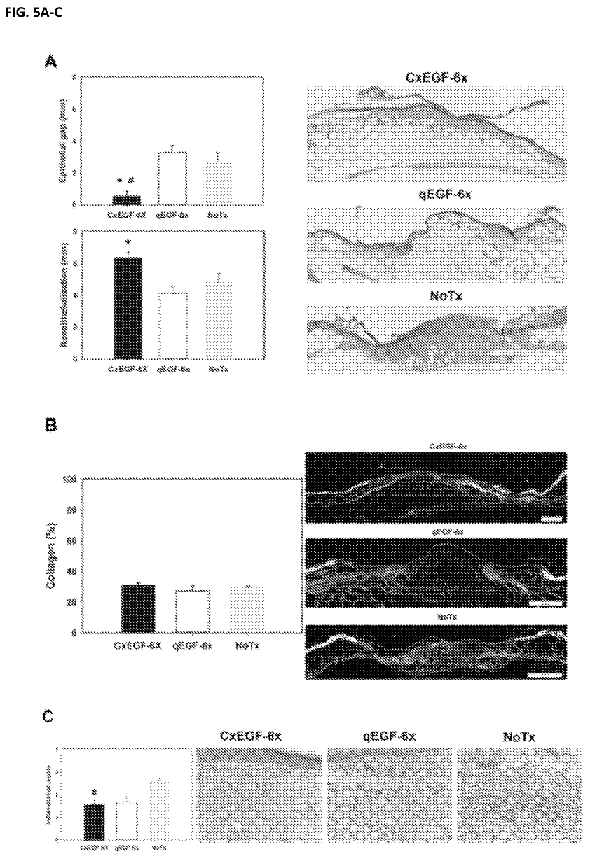

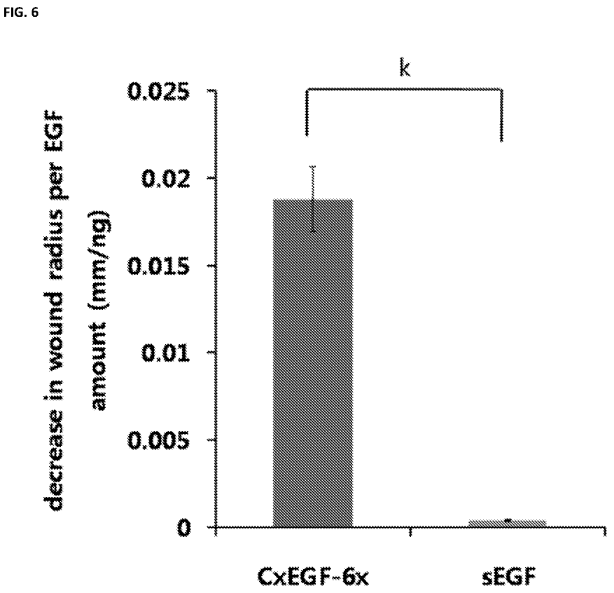

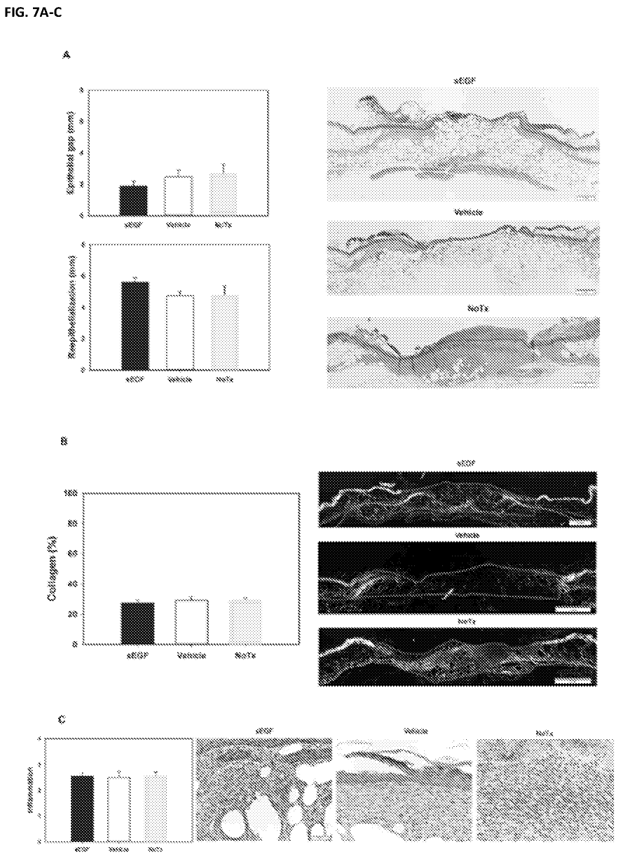

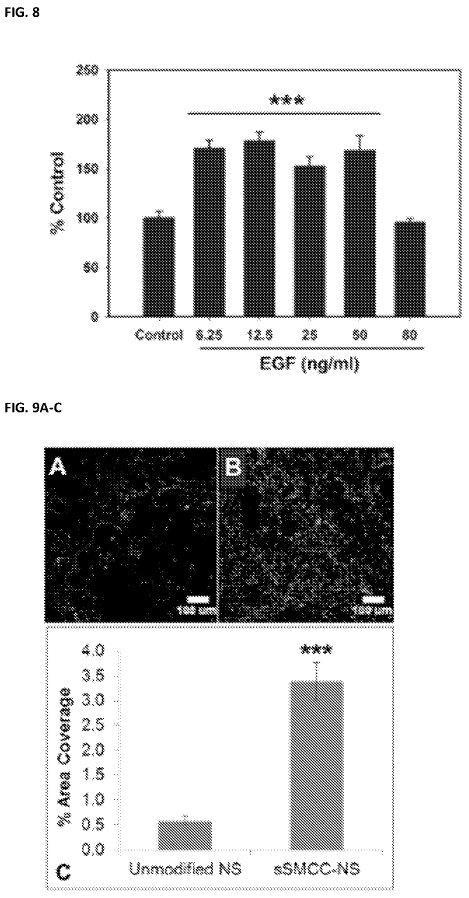

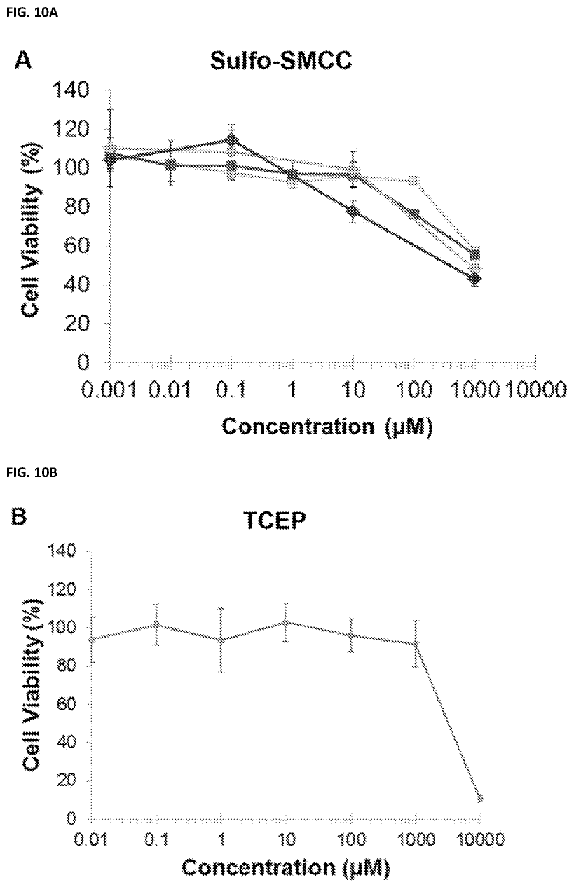

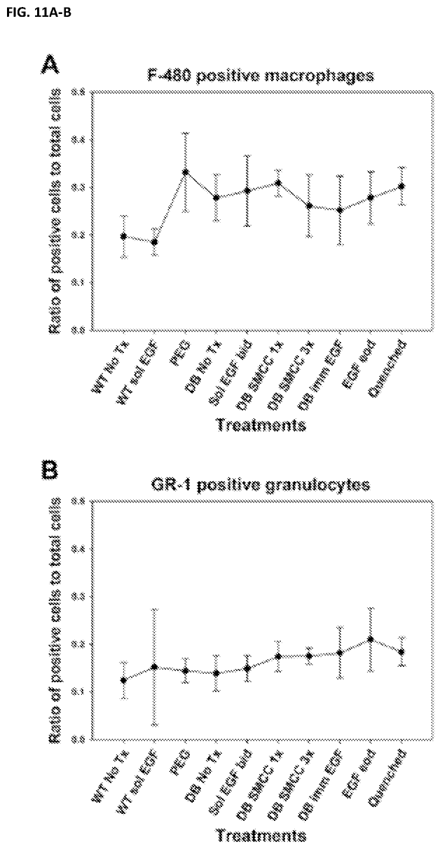

The feasibility to tether sulfo-SMCC modified nanospheres to graft tissue was first evaluated (FIG. 9). Full thickness splinted wounds (8 mm in diameter) were created on heterozygous (db+/−;BKS.Cg-m+/+Leprdb) mouse as described above. Wound surfaces were treated with a reducing agent (tris(2-carboxyethyl)phosphine; TCEP) to break the disulfide bonds. The crosslinkers and reducing agents were used at non-toxic doses (FIG. 10). This facilitated the exposed sulfhydryl groups for attachment to the malemide group from sSMCC modified EGF (here forth referred to as CxEGF; FIG. 3A). The fresh wound surface was treated with 10 μM TCEP (40 μl), following which sSMCC-EGF (30 ng in 30 μl of 5% PEG) was crosslinked onto the wound surfaces (denoted as CxEGF). Immediately after the treatment, wounds were covered with a semi-occlusive dressing to minimize scab formation (Park Shin Ae et al., 2014). Wound beds were harvested at various time points and the presence of EGF attached on the surfaces was determined by ELISA (FIG. 3B). Covalently tethering EGF to the wound surface resulted in sustained surface immobilization of the growth factor over 2 days. A gradual decrease in the amount of surface immobilized EGF was observed after 2 days. This apparent surface coverage (2 pg/mm², an equivalent of 1.99×10⁸ molecules/mm²) was observed to be 1/100th of a theoretical monolayer (2.19×10¹⁰ molecules/mm² equivalent to 213.6 pg/mm²) of EGF (Chai et al., 2000). We note that the number of disulfide bonds available for anchoring CxEGF is difficult to ascertain in vivo and may thus contribute to the partial surface coverage. Secondary validation of the linkage chemistry was done using an in vitro model of the surface chemistry using (3 mercaptopropyl)trimethoxysilane (MPTS) or trimethoxy(propyl)silane (TPS) and maleimide by a reduction in contact angle and increased thickness of deposition (FIG. 3D). The mercapto group on the MPTS simulated sulfhydryl groups present on a native wound bed, and TPS was devoid of any functional group simulating a passivated wound surface. The bioactivity of covalently immobilized CxEGF, on MPTS or TPS coated glass substrates was confirmed using an in vitro cell based bioassay (MTT) wherein cell proliferation significantly increased greater than 2-fold after 3 days of treatment (FIG. 3C). In fact, proliferation of cells cultured in 1% serum on surfaces with immobilized EGF were only 20% lesser than those observed when cells were cultured without exogenous EGF in the presence of 10% serum demonstrating potent bioactivity of immobilized EGF.

### Covalently Immobilized EGF Promotes Wound Healing In Vivo

Full thickness splinted wounds were created as previously described (Park S. A. et al., 2014), pre-treated with TCEP, and treated with either (i) soluble topical EGF, (ii) crosslinker control (SMCC), (iii) sSMCC modified EGF with its maleimide group quenched with β-mercaptoethanol (qEGF) or (iv) sSMCC modified EGF (CxEGF). Initial experiments were performed with immobilization performed only once (CxEGF-lx), immediately after wounding, and no significant effects on wound closure rate (reduction of wound radii) were observed (FIG. 3C, FIG. 7) despite documented retention of EGF for 2 days. Therefore, to prolong exposure to EGF, wounds were treated with CxEGF every other day (CxEGF-6x) or every 4 days (CxEGF-3x) after disulfide bonds were reduced using TCEP at each time point. This targeted delivery of growth factor resulted in significant acceleration of wound closure between 5 and 11 days after wounding when CxEGF was applied every other day in comparison with all other groups (FIG. 4A, B). Covalent application EGF every 4 days resulted in a similar decrease in wound radii as every other day treatments (FIG. 4C). That the increase in rate of re-epithelialization was attributed to covalent immobilization was verified by quenching the malemide groups of sSMCC-EGF with β-mercaptoethanol (qEGF). Indeed, qEGF treatment was ineffective in altering wound healing rates and was comparable to those of sEGF groups. Histological analyses demonstrated dramatic decreases in epithelial gap (FIG. 5A) after treatment with CxEGF-6X, with comparable collagen content (FIG. 5B) and reduction in inflammation (FIG. 5C) in comparison with qEGF or no treatment or soluble EGF.

Further studies failed to show a statistically significant differences in the numbers of F480 positive macrophages or GR1 positive granulocytes (FIG. 11). Of great relevance in the clinical use of highly expensive therapeutics such as growth factors is the greatly reduced amount of test article requires to achieve therapeutic benefit.

The total amount of EGF administered to the wound surface by covalent linkage of CxEGF-6X is calculated to be 180 ng, CxEGF-3X is calculated to be 90 ng, and for topical administration of sEGF 7200 ng was delivered over the 11 days. Yet, the rate of increased re-epithelialization was over 20 fold greater for the CxEGF-6X group than the sEGF group (FIG. 6).

### CONCLUSIONS

The above results point to a new and easily implemented approach for the delivery of therapeutic agents. Essentially the approach changes the microenvironment of the wound to promote wound closure rather than treating the wound with soluble agents or introducing macro-constructions of biomaterials. The results document the efficacy of using direct covalent immobilization to improve wound healing outcomes. We have demonstrated the efficacy of integrating EGF into the wound bed but the positive in vivo results suggest a broad application of this approach. The approach is generalizable and holds promise for integrating cytoactive factors, antimicrobials, analgesics, anesthetics and extracellular matrices to the patient's benefit. We note that this is approach has numerous advantages including (1) markedly decreased treatment frequency. This will reduce discomfort associated with frequent bandage changes and increase ‘patient’ compliance. It is even foreseeable that protocols will be simplified to enable in home treatment. (2) Decreased cost of (sometimes very costly) agents used due to markedly decreased total amount of agent expended. (3) Ability to utilize a wide array of commercially available agents. (4) Decreased adverse events predicted as the total amount of systemic exposure is much less than with frequent topical applications.

### REFERENCES

Abramovitch R, Marikovsky M, Meir G, Neeman M. Stimulation of tumour growth by wound-derived growth factors. Br J Cancer 1999; 79(9-10):1392-8.
American Diabetes A. Economic costs of diabetes in the U.S. in 2012. Diabetes Care 306 2013; 36(4):1033-46.
Barrientos S, Stojadinovic O, Golinko M S, Brem H, Tomic-Canic M. PERSPECTIVE ARTICLE: Growth factors and cytokines in wound healing. Wound Repair Regen 2008; 16(5):585-601.
Bennett N T, Schultz G S. Growth-Factors and Wound-Healing—Biochemical-Properties of Growth-Factors and Their Receptors. Am J Surg 1993; 165(6):728-37.
Bennett N T, Schultz G S. Growth factors and wound healing: Part II. Role in normal and chronic wound healing. The American Journal of Surgery 1993; 166(1):74-81.
Blaker J J, Nazhat S N, Boccaccini A R. Development and characterisation of silver-doped bioactive glass-coated sutures for tissue engineering and wound healing applications. Biomaterials 2004; 25(7-8):1319-29.
Boateng J S, Pawar H V, Tetteh J. Polyox and carrageenan based composite film dressing containing anti-microbial and anti-inflammatory drugs for effective wound healing. Int J Pharm 2013; 441(1-2):181-91.
Chai J J, Li M, Huang B R, Luo Y, Luo M, Bi R C, et al. Crystallization and preliminary X-ray diffraction studies of human epidermal growth factor. Acta Crystallogr D Biol Crystallogr 2000; 56(Pt 1):62-3.
Chattopadhyay S, Guthrie K M, Teixeira L, Murphy C J, Dubielzig R R, McAnulty J F, et al. Anchoring a cytoactive factor in a wound bed promotes healing. J Tissue Eng Regen Med 2014:n/a-n/a. Journal of Investigative Dermatology Page 16 of 37
Falanga V. Wound healing and its impairment in the diabetic foot. The Lancet; 366(9498):1736-327 43.
Gipson I K, Inatomi T. Extracellular matrix and growth factors in corneal wound healing. Curr Opin Ophthalmol 1995; 6(4):3.
Gottrup F, Apelqvist J, Price P, Group EWMAPO. Outcomes in controlled and comparative studies on non-healing wounds: recommendations to improve the quality of evidence in wound management. J Wound Care 2010; 19(6):237-68.
Hynes R O. The extracellular matrix: not just pretty fibrils. Science 2009; 326(5957):1216-9.
Jain R, Agarwal A, Kierski P R, Schurr M J, Murphy C J, McAnulty J F, et al. The use of native chemical functional groups presented by wound beds for the covalent attachment of polymeric microcarriers of bioactive factors. Biomaterials 2013; 34(2):340-52.
Jost M, Kari C, Rodeck U. The EGF receptor—an essential regulator of multiple epidermal functions. Eur J Dermatol 2000; 10(7):505-10.
Lerman O Z, Galiano R D, Armour M, Levine J P, Gurtner G C. Cellular dysfunction in the diabetic fibroblast: impairment in migration, vascular endothelial growth factor production, and response to hypoxia. Am J Pathol 2003; 162(1):303-12.
Loot M A, Kenter S B, Au F L, van Galen W J, Middelkoop E, Bos J D, et al. Fibroblasts derived from chronic diabetic ulcers differ in their response to stimulation with EGF, IGF-I, bFGF and PDGF-AB compared to controls. Eur J Cell Biol 2002; 81(3):153-60.
Maderal A D, Vivas A C, Eaglstein W H, Kirsner R S. The FDA and designing clinical trials for chronic cutaneous ulcers. Semin Cell Dev Biol 2012; 23(9):993-9.
Marquez B L, Watts K S, Yokochi A, Roberts M A, Verdier-Pinard P, Jimenez J I, et al. Structure and absolute stereochemistry of hectochlorin, a potent stimulator of actin assembly. J Nat Prod 2002; 65(6):866-71.
Martino M M, Tortelli F, Mochizuki M, Traub S, Ben-David D, Kuhn G A, et al. Engineering the growth factor microenvironment with fibronectin domains to promote wound and bone tissue healing. Sci Transl Med 2011; 3(100):100ra89.
Masters K S. Covalent growth factor immobilization strategies for tissue repair and regeneration. Macromol Biosci 2011; 11(9):1149-63.
Park S A, Raghunathan V K, Shah N M, Teixeira L, Motta M J, Covert J, et al. PDGF-BB does not accelerate healing in diabetic mice with splinted skin wounds. PLoS One 2014; 9(8):e104447.
Park S A, Teixeira L B, Raghunathan V K, Covert J, Dubielzig R R, Isseroff R R, et al. Full-thickness splinted skin wound healing models in db/db and heterozygous mice: implications for wound healing impairment. Wound Repair Regen 2014; 22(3):368-80.
Pawar H V, Boateng J S, Ayensu I, Tetteh J. Multifunctional medicated lyophilised wafer dressing for effective chronic wound healing. J Pharm Sci 2014; 103(6):1720-33.
Pawar H V, Tetteh J, Boateng J S. Preparation, optimisation and characterisation of novel wound healing film dressings loaded with streptomycin and diclofenac. Colloids Surf B Biointerfaces 2013; 102:102-10.
Raffetto J D, Mendez M V, Marien B J, Byers H R, Phillips T J, Park H Y, et al. Changes in cellular motility and cytoskeletal actin in fibroblasts from patients with chronic venous insufficiency and in neonatal fibroblasts in the presence of chronic wound fluid. J Vasc Surg 2001; 33(6):1233-41.
Raghunathan V K, Devey M, Hawkins S, Hails L, Davis S A, Mann S, et al. Influence of particle size and reactive oxygen species on cobalt chrome nanoparticle-mediated genotoxicity. Biomaterials 2013; 34(14):3559-70.
Rennekampff H O, Hansbrough J F, Kiessig V, Dore C, Sticherling M, Schroder J M. Bioactive 377 interleukin-8 is expressed in wounds and enhances wound healing. J Surg Res 378 2000; 93(1):41-54.
Robson M C, Maggi S P, Smith P D, Wassermann R J, Mosiello G C, Hill D P, et al. Original articles: ease of wound closure as an endpoint of treatment efficacy. Wound Repair Regen 1999; 7(2):90-6.
Schneider A, Wang X Y, Kaplan D L, Garlick J A, Egles C. Biofunctionalized electrospun silk mats as a topical bioactive dressing for accelerated wound healing. Acta Biomater 2009; 5(7):2570-8.
Seidman C, Raffetto J D, Marien B, Kroon C, Seah C C, Menzoian J O. bFGF-induced alterations in cellular markers of senescence in growth-rescued fibroblasts from chronic venous ulcer and venous reflux patients. Ann Vasc Surg 2003; 17(3):239-44.
Sen C K, Gordillo G M, Roy S, Kirsner R, Lambert L, Hunt T K, et al. Human skin wounds: a major and snowballing threat to public health and the economy. Wound Repair Regen 2009; 17(6):763-71.
Stefonek-Puccinelli T J, Masters K S. Co-immobilization of gradient-patterned growth factors for directed cell migration. Ann Biomed Eng 2008; 36(12):2121-33.
Stefonek T J, Masters K S. Immobilized gradients of epidermal growth factor promote accelerated and directed keratinocyte migration. Wound Repair Regen 2007; 15(6):847-55.
Stromberg K, Chapekar M S, Goldman B A, Chambers W A, Cavagnaro J A. Regulatory concerns in the development of topical recombinant ophthalmic and cutaneous wound healing biologics. Wound Repair Regen 1994; 2(3):155-64.
Vasquez R, Marien B J, Gram C, Goodwin D G, Menzoian J O, Raffetto J D. Proliferative capacity of venous ulcer wound fibroblasts in the presence of platelet-derived growth factor. Vasc Endovascular Surg 2004; 38(4):355-60.
Wieman T J, Smiell J M, Su Y. Efficacy and safety of a topical gel formulation of recombinant human platelet-derived growth factor-BB (becaplermin) in patients with chronic neuropathic diabetic ulcers. A phase III randomized placebo-controlled double-blind study. Diabetes Care 1998; 21(5):822-7.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

What is claimed is:

1. A growth factor (GF) conjugate comprising a GF molecule covalently modified with a chemical moiety comprising a reactive functionality.

2. The conjugate of claim 1, wherein the GF molecule is recombinant.

3. The conjugate of claim 1, wherein the GF molecule is a human GF molecule.

4. The conjugate of claim 1, wherein the GF molecule is a non-human EGF molecule.

5-9. (canceled)

10. The conjugate of claim 1, wherein the GF molecule comprises a genetically encoded aldehyde tag.

11. The conjugate of claim 10, wherein the genetically encoded aldehyde tag has a sequence selected from the group consisting of LCTPSRAALLTGR (ALD₁₃) and LCTPSR (ALD₆).

12. The conjugate of claim 1, wherein the GF molecule is selected from the group consisting of an Epidermal Growth Factor (EGF) molecule, a Vascular Endothelial Growth Factor (VEGF) molecule, a Platelet-Derived Growth Factor (PDGF) molecule, a Fibroblast Growth Factor (FGF) molecule, a Keratinocyte Growth Factor (KGF) molecule, a Transforming Growth Factor Beta (TGF-β) molecule, a Granulocyte-Macrophage Colony-Stimulating Factor (GM-CSF) molecule, an Insulin-Like Growth Factor I (IGF-I) molecule, an Insulin-Like Growth Factor II (IGF-II) molecule, Nerve Growth Factor (NGF)

13. The conjugate claim 1, wherein the reactive functionality is selected from the group consisting of a sulfhydryl reactive functionality, an amine reactive functionality, a carbonyl reactive functionality, and a carboxyl reactive functionality.

14. The conjugate of claim 1, wherein the chemical moiety is covalently attached to the GF molecule at a chemical group in the GF molecule selected from the group consisting of an amine group, a sulfhydryl group, a carbonyl group, and a carboxyl group.

15. A method of treating chronic wound in a subject in need thereof comprising:
applying a conjugate of claim 1 to the chronic wound under conditions such that the conjugate covalently binds to a target group in the wound that is reactive with the reactive functionality.

16. The method of claim 15, wherein the subject is a human subject.

17. The method of claim 15, wherein the subject is a non-human subject.

18. The method of claim 15, wherein the chronic would is selected form the group consisting diabetic ulcers, pressure ulcers, traumatic ulcers, venous stasis ulcers, venous leg ulcers, and arterial ulcers.

19. The method of claim 15, wherein the chronic wound has a wound surface and the conjugate is applied to the wound surface in an amount sufficient to cover the wound surface with from 0.1 to 20 pg of the conjugate per mm² of the wound surface.

20. The method of claim 15, wherein the conjugate is applied every 24 to 72 hours.

21. The method of claim 15, wherein the conjugate is applied every 36 to 60 hours.

22-23. (canceled)

24. The method of claim 15, wherein the conjugate is applied until more than 90% of the wound is re-epithelialized.

25. (canceled)

26. The method of claim 15, wherein the conjugate is applied to the wound from about 3 to about 30 times.

27-28. (canceled)

29. The method of claim 15, wherein the conjugate is provided in a polymeric matrix.

30-32. (canceled)

33. A delivery device comprising the conjugate of claim 1 in a polymeric matrix.
